(19) United States
(12) Patent Application Publication
(10) Pub. No.: US 2024/0081857 A1
(43) Pub. Date: Mar. 14, 2024

(54) RECANALIZATION DEVICES AND ASSOCIATED SYSTEMS AND METHODS

(71) Applicant: Inari Medical, Inc., Irvine, CA (US)

(72) Inventors: Hieu Luong, Westminster, CA (US); Taylor Scheinblum, Newport Beach, CA (US); John Cole Thress, Oceanside, CA (US); Derek Hauschka, Orange, CA (US); Jacob Taylor, Stockton, CA (US)

(21) Appl. No.: 18/467,500

(22) Filed: Sep. 14, 2023

Related U.S. Application Data
(60) Provisional application No. 63/375,682, filed on Sep. 14, 2022.

(51) Int. Cl. A61B 17/3207

(52) U.S. Cl. CPC A61B 17/320758; A61B 2017/320052

(57) ABSTRACT

Provided herein is a recanalization system including (i) a handle located at a proximal end of the system, (ii) a first elongate member, (iii) a second elongate member, the first and second elongate members being movable relative to each other elongate member, and (iv) a recanalization device located at a distal end of the system. The recanalization device includes (i) a first end portion coupled to the first elongate member, (ii) a second end portion coupled to the second elongate member, and (iii) at least one strut extending between the first and second end portions. The recanalization device is configured to be (i) selectively radially expanded and compressed and (ii) axially moved relative to the first and second elongate members.

### CROSS-REFERENCE TO RELATED APPLICATIONS

This application claims priority to U.S. Provisional Patent Application Ser. No. 63/375,682, filed on Sep. 14, 2022, the disclosure of which is hereby incorporated by reference in its entirety.

### FIELD OF THE DISCLOSURE

The field of the disclosure relates generally to recanalization devices and, more particularly, to recanalization devices configured to recanalize occluded vessel segments, particularly chronically occluded vessel segments.

### BACKGROUND

Post-Thrombotic Syndrome (PTS) is a serious condition that may occur after a patient has a blood clot (e.g., deep vein thrombosis or DVT) and includes constant pain, edema, and other symptoms that arise from clot material still being present within the vasculature of the patient. Further, the longer clot material is present within the vasculature of the patient, the more the clot material hardens, making it difficult for therapeutics and device to clear the clot material. This hardening may further exacerbate the symptoms of PTS. Accordingly, the present treatments for chronically occluded vasculature include symptom management, endophlebectomy, or complete vein removal (an invasive procedure).

Current systems/devices for clearing clot material limit a user's control over the clearing process. For example, U.S. Pat. No. 10,779,852 (hereinafter “the '852 patent”) and U.S. Pat. No. 11,406,418 (hereinafter “the '418 patent) describe a current thrombectomy device. The thrombectomy device includes a self-adjusting cage where a resistance mechanism is adjusted without any user input other than the actions of the user to move the device along a vessel. Therefore, the diameter of the self-adjusting cage is dependent on the diameter of the vessel.

Based on the foregoing, there is a need for a recanalization system which provides a user increased control over the expansion and contraction of the recanalization device.

### BRIEF SUMMARY

In one aspect, a recanalization system including a handle located at the proximal end of the system, a first elongate member, a second elongate member, and a recanalization device located at a distal end of the system is provided. The handle includes an actuator. The first elongate member is movable relative to the second elongate member. The recanalization device includes a first end portion coupled to the first elongate member, a second portion coupled to the second elongate member, and at least one strut extending between the first and second end portions. The actuator is configured to move the first end portion toward the second end portion to expand each of the at least one struts, and the recanalization device is configured to move relative to the first and second elongate members.

In another aspect, a method for recanalization is provided. The method includes (i) advancing a plurality of elongate members of a recanalization system intravascularly to a target location within a patient, (ii) radially expanding a recanalization device of the recanalization system to engage with the target location, and (iii) axially moving the recanalization device to clear clotted material at the target location; and (iv) withdrawing the plurality of elongate members of the recanalization system from the patient.

In yet another aspect, a recanalization device is provided. The recanalization device includes (i) a first end and a second end, and a center between the first and second ends, the first and second ends being spaced apart along an axis, the first and second ends being movable toward and away from the center of the recanalization device, (ii) at least one strut member extending away from each of the first and second ends, and terminating at an apex member, and (iii) a diamond cell strut extending between and integral with two apex members. Each diamond cell strut includes (i) opposed outwardly directed edges and (ii) a number of teeth located along each edge.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

As used herein, “a”, “an”, and “the” refer to both singular and plural referents unless the context clearly dictates otherwise.

As used herein, the term “or”, including the claims, is used to mean “and/or” unless explicitly indicated to refer to alternatives only or to refer to the alternatives that are mutually exclusive.

As used herein, the term “about” refers to a measurable value such as a parameter, an amount, a temporal duration, and the like and is meant to include variations of +/−15% or less, preferably variations of +/−10% or less, more preferably variations of +/−5% or less, even more preferably variations of +/−1% or less, and still more preferably variations of +/−0.1% or less of and from the particularly recited value, in so far as such variations are appropriate to perform in the invention described herein. Furthermore, it is also to be understood that the value to which the modifier “about” refers is itself specifically disclosed herein.

As used herein, spatially relative terms, such as “beneath”, “below”, “lower”, “above”, “upper”, “front”, “back”, “side”, “left”, “right”, “rear”, and the like, are used for ease of description to describe one element or feature's relationship to another element(s) or feature(s). It is further understood that the terms “front”, “back”, “left”, and “right” are not intended to be limiting and are intended to be interchangeable, where appropriate. Further, it should be noted that the terms “first,” “second,” and the like herein do not denote any order, quantity, or relative importance, but rather are used to distinguish one element from another.

As used herein, the terms “comprise(s)”, “comprising”, and the like, specify the presence of stated features, integers, steps, operations, elements, and/or components, but do not preclude the presence or addition of one or more other features, integers, steps, operations, elements, components, and/or groups thereof.

As used herein, the terms “configure(s)”, “configuring”, and the like, refer to the capability of a component and/or assembly, but do not preclude the presence or addition of other capabilities, features, components, elements, operations, and any combinations thereof.

Chemical compounds are described using standard nomenclature. For example, any position not substituted by any indicated group is understood to have its valency filled by a bond as indicated, or a by hydrogen atom.

All ranges disclosed herein are inclusive of the endpoints, and the endpoints are independently combinable with each other. Each range disclosed herein constitutes a disclosure of any point or sub-range lying within the disclosed range.

All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention or any embodiments unless otherwise claimed.

Any combination or permutation of features, functions and/or embodiments as disclosed herein is envisioned. Additional advantageous features, functions and applications of the disclosed systems, methods and assemblies of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale. Corresponding reference characters indicate corresponding parts throughout the several views of the drawings.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIG. 1A is a side perspective view of a recanalization system in accordance with embodiments of the present disclosure;

FIG. 1B is a side cross-sectional view of the recanalization system of FIG. 1A in accordance with embodiments of the present disclosure;

FIG. 2A is a side view of a recanalization system in accordance with embodiments of the present disclosure;

FIG. 2B is a side cross-sectional view of the recanalization system of FIG. 2A with a recanalization device in a first position, in accordance with embodiments of the present disclosure;

FIG. 2C is a side cross-sectional view of the recanalization system of FIG. 2A with a recanalization device in a second position, in accordance with embodiments of the present disclosure;

FIG. 2D is a magnified view of the trigger of the recanalization system of FIG. 2B in accordance with embodiments of the present disclosure;

FIG. 2E is a magnified view of the leadscrew of the recanalization system of FIG. 2B in accordance with embodiments of the present disclosure;

FIG. 2F is a detailed view of the biasing member of the recanalization system of FIG. 2B in accordance with embodiments of the present disclosure;

FIG. 3A is a perspective view of a recanalization system in accordance with embodiments of the present disclosure;

FIG. 3B is a magnified perspective view of the recanalization system of FIG. 3A in accordance with embodiments of the present disclosure;

FIG. 3C is a perspective cross-sectional view of the recanalization system of FIG. 3A in accordance with embodiments of the present disclosure;

FIG. 3D is a detailed view of the biasing member of the recanalization system of FIG. 3C in accordance with embodiments of the present disclosure;

FIG. 4 is a side perspective view of a recanalization device of the recanalization system of FIGS. 1A-3D in accordance with embodiments of the present disclosure;

FIG. 5A is a side perspective view of a recanalization device of the recanalization system of FIGS. 1A-3D in accordance with embodiments of the present disclosure;

FIG. 5B is a partial, magnified cross-sectional view of the recanalization device of FIG. 5A in accordance with embodiments of the present disclosure;

FIG. 6 is a partial, magnified perspective view of the recanalization device of FIG. 5A in accordance with embodiments of the present disclosure;

FIG. 7A is a partial, magnified perspective view of the recanalization device of FIG. 5A in accordance with embodiments of the present disclosure;

FIG. 7B is a partial, magnified cross-sectional view of the recanalization device of FIG. 7A in accordance with embodiments of the present disclosure;

FIG. 8A is a perspective view of a portion of a portion of a diamond cell strut of recanalization device of FIGS. 4-7B in accordance with embodiments of the present disclosure;

FIG. 8B is a perspective view of a portion of another diamond cell strut of the recanalization device of FIGS. 4-7B in accordance with embodiments of the present disclosure;

FIG. 9A is a side perspective view of a recanalization device of the recanalization system of FIGS. 1A-3D in a first position in accordance with embodiments of the present disclosure;

FIG. 9B is a side perspective view of a recanalization device of the recanalization system of FIGS. 1A-3D in a second position in accordance with embodiments of the present disclosure;

FIG. 10 is a diagram of an exemplary method associated with the recanalization system of FIGS. 1A-3D, in accordance with embodiments of the present disclosure;

FIG. 11 is a side perspective view of an embodiment of an elongate member associated with the recanalization system of FIGS. 1A-3D in accordance with embodiments of the present disclosure;

FIG. 12 is a side perspective view of an embodiment of a recanalization device associated with the recanalization system of FIGS. 1A-3D in accordance with embodiments of the present disclosure; and

FIG. 13 is a perspective view of an embodiment of a recanalization device associated with the recanalization system of FIGS. 1A-3D in accordance with embodiments of the present disclosure.

### DETAILED DESCRIPTION

The present disclosure is generally directed to recanalization systems and devices for recanalizing vein segments (e.g., severely chronically occluded vein segments including iliofemoral vein segments) of a patient, such as a patient suffering from Post-Thrombotic Syndrome (PTS), and associated systems and methods. The recanalization systems and devices may be used by a user (e.g., a physician, clinician, or other trained provider) to treat the occluded vein segments of the user.

In some embodiments described herein, a recanalization system for engaging and clearing clot material from chronically occluded vasculature of patients includes (i) a handle, (ii) a recanalization device, and (iii) a plurality of elongate members. The recanalization device is configured to be deployed within the vasculature of the patient, and includes a first end portion (e.g., a distal portion), a second end portion (e.g., a proximal portion), and at least one strut extending between the first and second end portions.

When expanded within the vasculature, the recanalization device, and more specifically, the at least one strut, can be (i) axially translated and/or (ii) rotated within the vasculature to engage with, and clear the clotted material. In some embodiments, the at least one strut includes teeth and/or edge modifications that are specifically configured to engage with aged, clotted material to break up the material without the need for a collection bag or aspiration device to remove the material from the patient.

The devices, systems, and methods described herein include technical benefits over known devices, systems, and methods. Specifically, the devices, systems, and methods described herein include at least the technical benefits of: (i) control of the diameter defined by the struts of the recanalization device for selectively applying a radial force and enabling progressive treatment, (ii) the struts of the recanalization device having higher radial force and sharp and/or serrated elements for better clot engagement, (iii) at least two independent elongate members (e.g., mobile shafts or catheters) for ease of use and rapid element contraction and expansion, (iv) 360° circumferential treatment for more effective clot removal, and (v) dual elongate members formed of solid nitinol hypotubes for flexibility, pushability, torquability, and kink resistance.

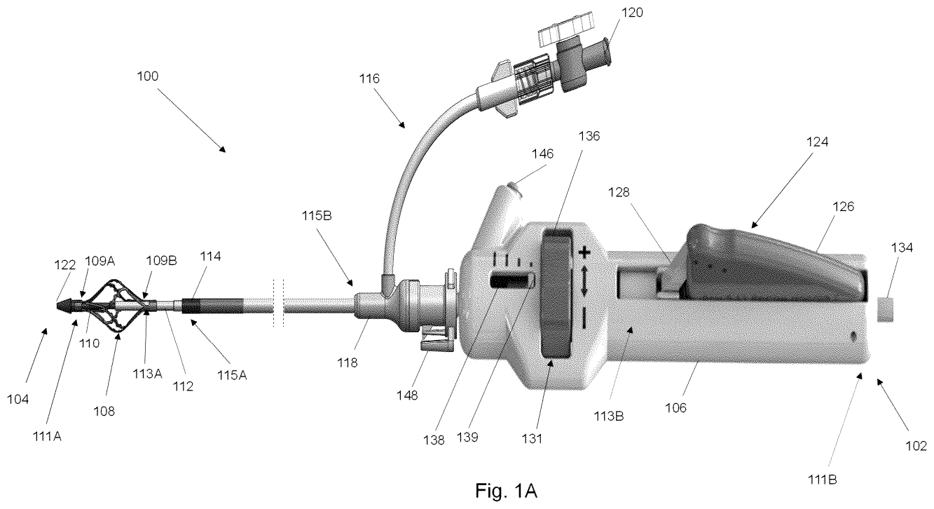

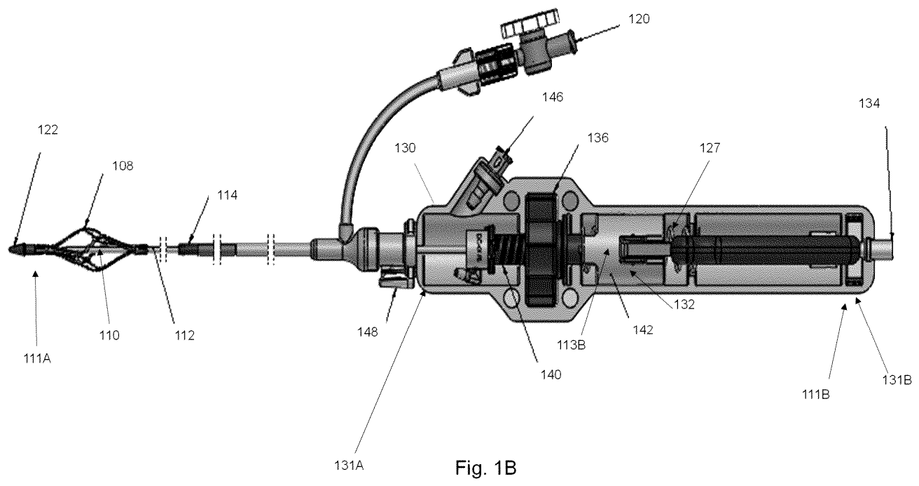

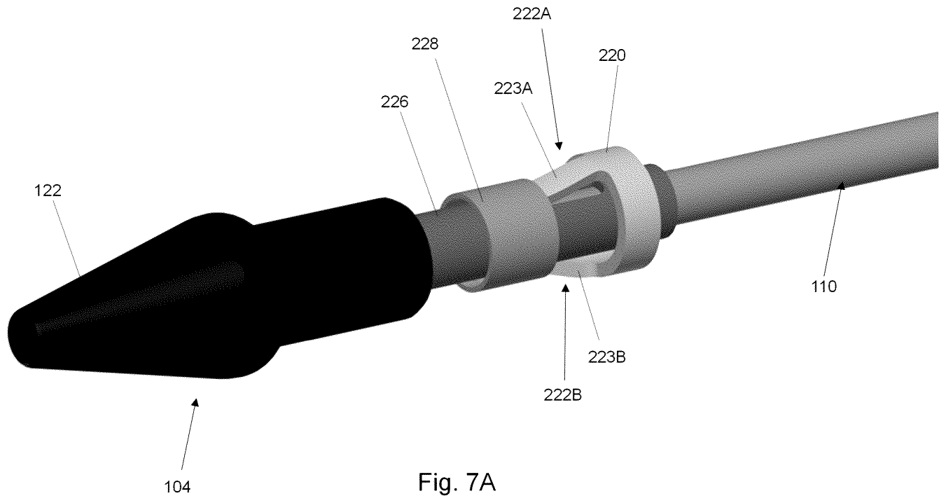

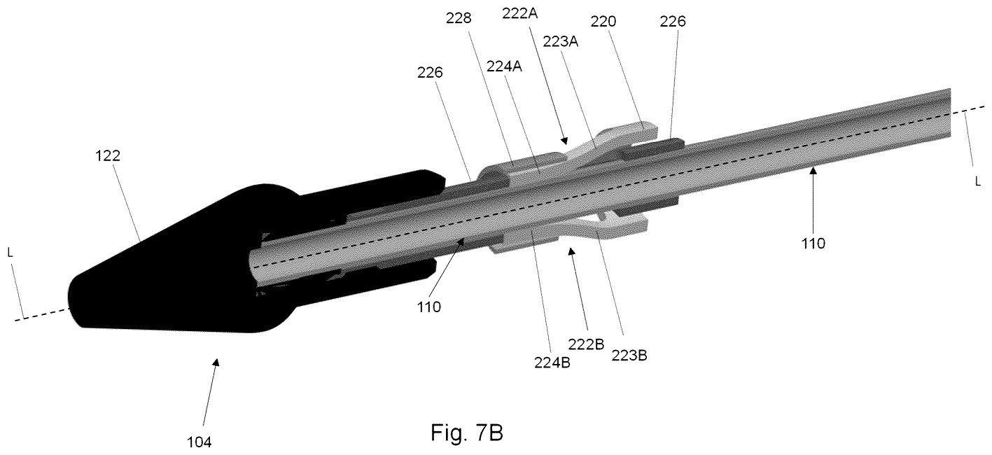

Certain details are set forth in the following description of FIGS. 1A-13 to provide a thorough understanding of various embodiments of the present technology. In other instances, well-known structures, materials, operations, and/or systems often associated with intravascular procedures, stents, clot and occluded vein segment removal procedures, catheters, and the like are not shown or described in detail in the following disclosure to avoid unnecessarily obscuring the description of the various embodiments of the disclosure. Those of ordinary skill in the art will recognize, however, that the present disclosure can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the disclosure. Indeed, certain terms may even be emphasized below; however, any terminology intended to be interpreted in any restricted manner will be overtly and specifically defined as such in the present section.

The accompanying Figures depict embodiments of the present disclosure and are not intended to be limiting of its scope unless expressly indicated. The sizes of various depicted elements are not necessarily drawn to scale, and these various elements may be enlarged to improve legibility. Component details may be represented in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present disclosure can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present disclosure can be used in any orientation suitable to the user.

Referring to FIGS. 1A-3D, in the illustrated embodiment, the system 100 extends from a proximal end 102 to a distal end 104. The proximal end 102 includes a handle 106 operably coupled to a recanalization device 108, located at the distal end 104, via an inner elongate member 110 (also referred to herein as a “first elongate member”) and a middle elongate member 112 (also referred to herein as a “second elongate member”). The inner elongate member 110 can extend through a lumen of the middle elongate member 112 such that the inner and middle elongate members 110, 112 (collectively “elongate members 110, 112”) are coaxial. The system 100 further includes an outer elongate member 114 (e.g., an aspirational catheter, a guide catheter or delivery catheter), and the elongate members 110, 112 at least partially extend through the outer elongate member 114 such that the inner, middle, and outer elongate members 110, 112, 114 (collectively “elongate members 110, 112, 114”) are coaxial (or nearly coaxial) relative to longitudinal axis (L). In other embodiments, the elongate members 110, 112 are not coaxial and may instead extend side-by-side and/or through separate lumens of the outer elongate member 114.

In the exemplary embodiment, the elongate members 110, 112 include hypotubes and are formed of nitinol to improve system 100 responsiveness, flexibility, kink resistance, and pushability. Further, in an exemplary embodiment, the outer elongate member 114 includes a braid/coil reflow construction with a low crossing profile. In other embodiments, the elongate members 110, 112, 114 may include catheters, tubes (e.g., hypotubes and laser-cut hypotubes), cables (e.g., helical hollow strand cables) sheathes, shafts (e.g., torque shafts), and/or the like, and the elongate members 110, 112, 114 may be formed from metal (e.g., stainless steel, nitinol, cobalt chrome), braid/coil reflow constructions, plastic, fluoropolymers (e.g., polytetrafluoroethylene (PTFE)), polymers, and/or other suitable materials.

The outer elongate member 114 includes a distal portion 115A and a proximal portion 115B. The proximal portion 115B may be directly or indirectly coupled to a tubing assembly 116 (e.g., including one or more tubes, fluid control devices, and/or the like) via a valve device 118. Valve device may include a disconnect 148 for the outer elongate member 114. Specifically, the disconnect 148 is configured to allow a user to quickly disconnect the outer elongate member 114 from the handle 106. In some embodiments, the tubing assembly 116 can be used to flush the lumen of the outer elongate member 114 through a side port 120. In some embodiments, the handle 106 is movable (translatable, rotatable) relative to the valve device 118 and the outer elongate member 114.

In the illustrated embodiment of FIGS. 1A-3D, the handle 106 has been advanced toward the valve device 118 such that the recanalization device 108 extends from the distal portion 115A of the outer elongate member 114. In some embodiments, the handle 106 can be retracted away from the outer elongate member 114 and/or the outer elongate member 114 can be advanced away from the handle 106 such that the recanalization device 108 is captured/positioned within the outer elongate member 114. When the recanalization device 108 is captured within the lumen of the outer elongate member 114, the recanalization device 108 is in a radially compressed state. In some embodiments, the recanalization device 108 can be positioned within the outer elongate member 114 during delivery of the system 100 through the vasculature of a patient.

In the illustrated embodiment, the system 100 includes a tip 122 located at the distal end 104 of the system 100 and may be coupled, either directly or indirectly, to the recanalization device 108 and/or the inner elongate member 110. The tip 122 may be removably attached, either directly or indirectly, to the recanalization device 108 and/or the inner elongate member 110. In some instances, the tip 122 may be semi-permanently or permanently attached, either directly or indirectly, to the recanalization device 108 and/or the inner elongate member 110. The tip 122 may have an atraumatic shape configured to minimize or even prevent damage to the vasculature (e.g., sized and shaped as a nosecone) of a patient as the system 100 is advanced therethrough, and the tip 122 may be formed of a polymer. In other embodiments, the tip 122 may be made from a material other than a polymer and, may have shapes/configurations other than as described, or may be omitted entirely. In some embodiments, when the recanalization device 108 is constrained within the outer elongate member 114, the tip 122 may engage the distal portion 115A of the outer elongate member 114 and may be shaped and sized to seal the lumen of the outer elongate member 114. In some embodiments, the tip 122 and the inner elongate member 110 can define a lumen 125 configured (e.g., sized and shaped) to receive a guidewire (not shown) therethrough. The system 100 can be advanceable/trackable over the guidewire.

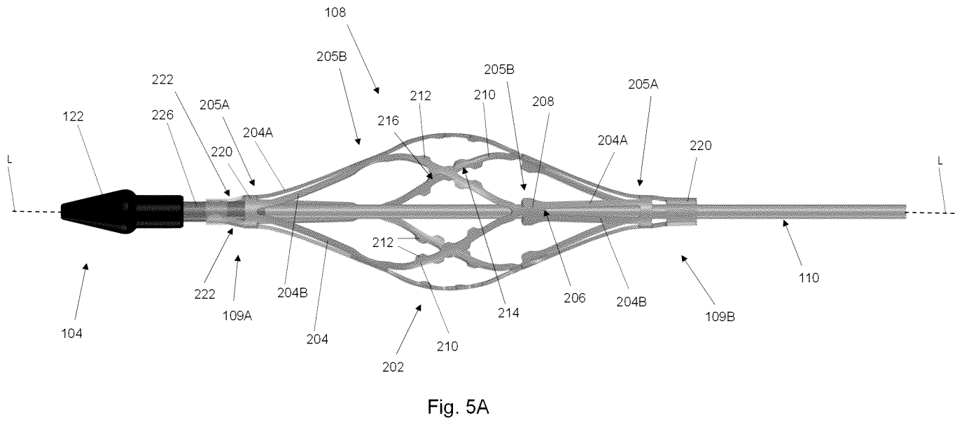

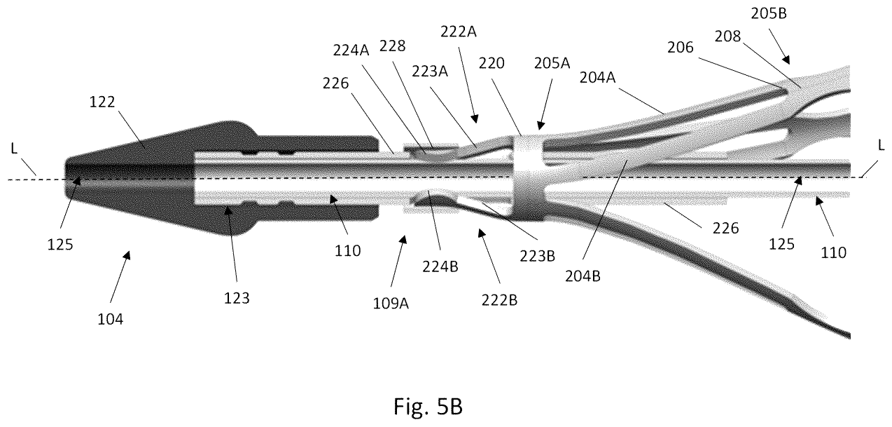

As shown in FIG. 5B, tip 122 may include a proximal cavity 123 configured to directly or indirectly engage with one or more elongate members 110, 112, 114 and/or recanalization device 108. Tip 122 may be directly or indirectly mounted with respect to inner elongate member 110 and/or recanalization device 108 using adhesive, welding, fasteners, crimping, press fitting, and combinations thereof. Tip 122 may define a lumen 125 configured (e.g., sized and shaped) to receive a guidewire (not shown) therethrough. Lumen 125 may extend longitudinally through at least inner elongate member 110 and tip 122.

System 100 may include extension member 226 which may directly or indirectly engage with at least inner elongate member 110 and/or tip 122. Extension member 226 may be removably attached to at least one of inner elongate member 110 and tip 122. In some instances, extension member 226 may be semi-permanently (or permanently) attached to at least one of inner elongate member 110 and tip 122. Extension member 226 may be in direct or indirect contact with recanalization device 108. Extension member 226 may define a sidewall created by a through hole extending longitudinally from the proximal end to the distal end.

Extension member 226 may directly or indirectly engage with recanalization device 108. Extension member 226 may define one or more features configured to engage with recanalization device 108. For example, extension member 226 may define one or more openings (not shown). The one or more openings may be circumferentially positioned about an outer surface of extension member 226. The one or more openings may extend at least partially through the exterior surface of a sidewall of the extension member 226. Recanalization device 108 may directly or indirectly engage with one or more openings of extension member 226.

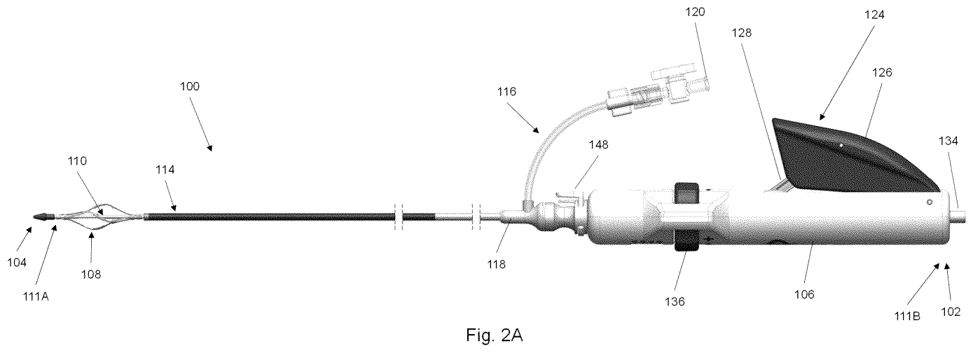

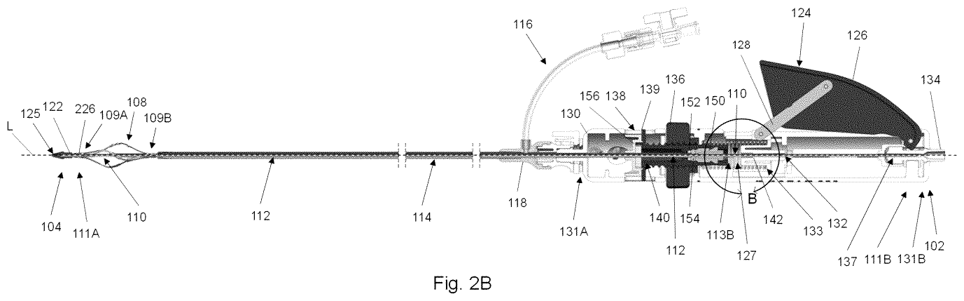

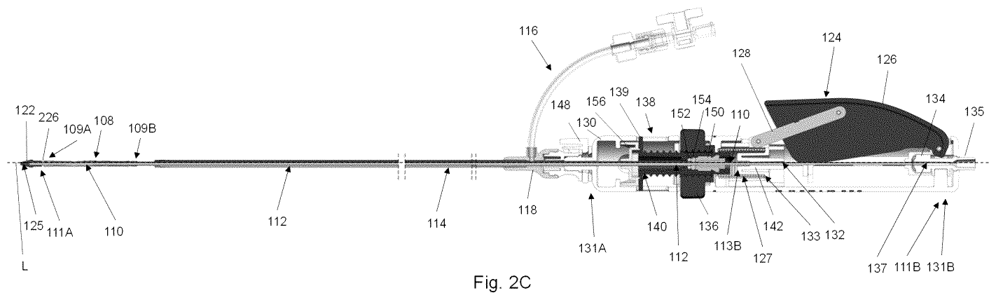

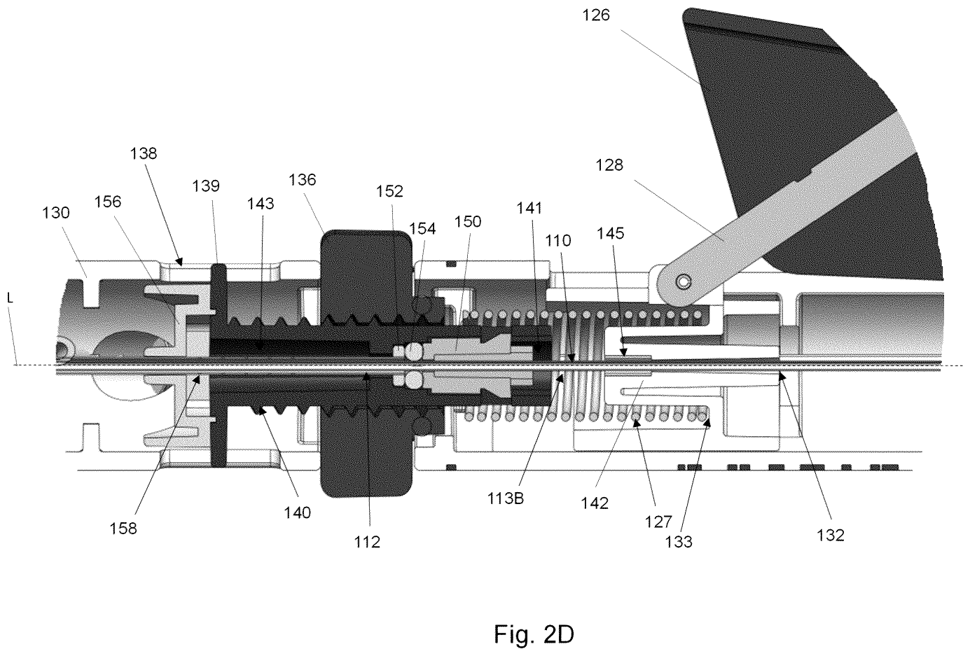

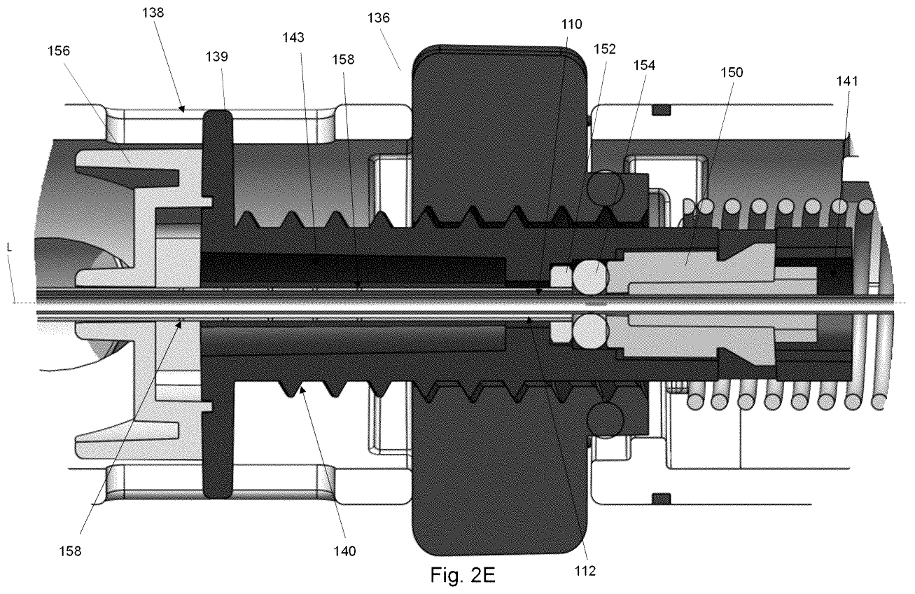

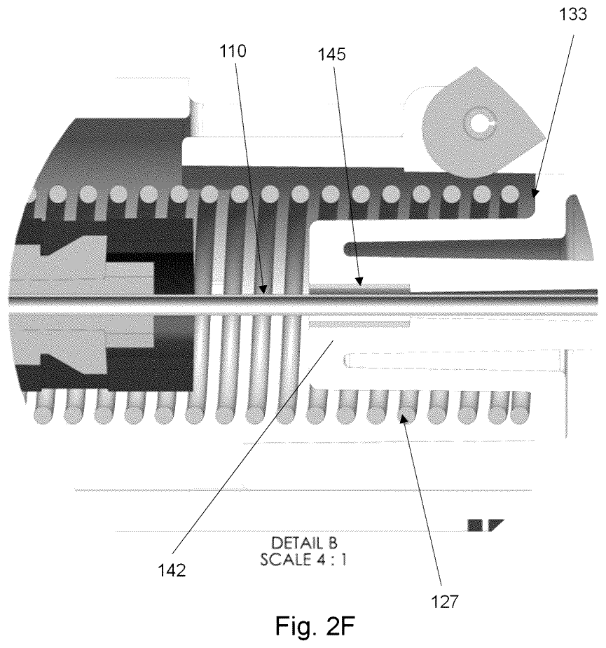

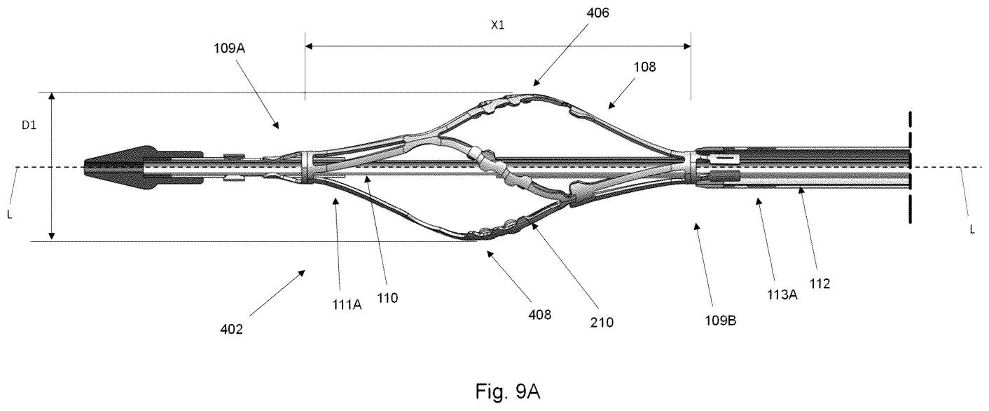

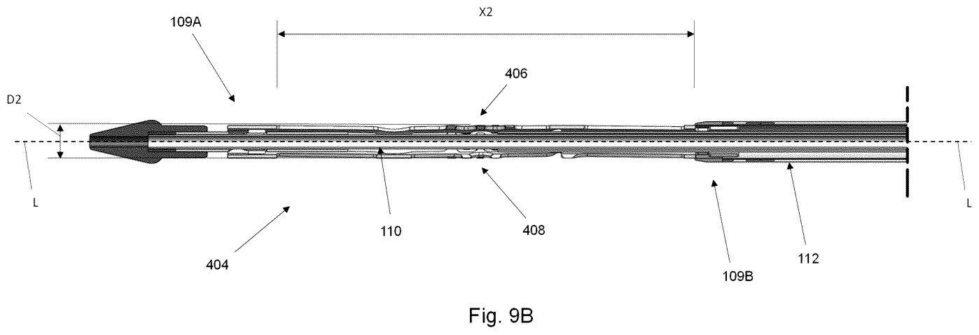

In an illustrated embodiment, the recanalization device 108 includes a distal portion 109A and a proximal portion 109B, the inner elongate member 110 includes a distal portion 111A and a proximal portion 111B (see, e.g., FIGS. 1A-2C), and the middle elongate member 112 includes a distal portion 113A and a proximal portion 113B (see, e.g., FIG. 1B). The distal portion 111A of the inner elongate member 110 is coupled to the distal portion 109A of the recanalization device 108. Similarly, the distal portion 113A of the middle elongate member 112 is coupled to the proximal portion 109B of the recanalization device 108. Accordingly, as described in further detail below, especially with respect to FIGS. 9A and 9B, relative movement of the elongate members 110, 112 can longitudinally shorten/lengthen and radially expand/compress the recanalization device 108 via the relative movement of the respective distal and proximal portions 109A, 109B of the recanalization device. The distal and proximal portions 109A, 109B of the recanalization device 108 can be identical and can have a cylindrical or hub-like shape. In some embodiments, the distal and proximal portions 109A, 109B are secured to the elongate members 110, 112, respectively, via adhesive (e.g., glue bonds), welding, fasteners, crimping and/or the like. For example, in some embodiments, one or both of the distal and proximal portions 109A, 109B of the recanalization device 108 can be directly welded to the elongate members 110, 112.

As illustrated in FIGS. 1A-3D the handle 106 includes a housing 130 having a distal portion 131A and a proximal portion 131B and defining an internal chamber or lumen 132. For clarity, housing 130 is shown as partially-cut away in FIG. 1B and as a cross-section in FIGS. 2B-2D. The proximal portions 111B, 113B of the elongate members 110, 112, respectively, extend through at least a portion of the housing 130.

In some embodiments, the handle 106 includes a port housing 134 operably (e.g., fluidly) coupled to the lumen of the inner elongate member 110 and/or the middle elongate member 112. Port housing 134 may extend outwardly from handle 106 and define port 135 which may be operably (e.g., fluidly) coupled to the lumen of the inner elongate member 110 and/or the middle elongate member 112. Port housing 134 may define lumen 137 that extends between the proximal portion and the distal portion of port housing 134, including port 135. Lumen 137 may be configured to directly or indirectly engage with inner elongate member 110. In some instances, inner elongate member 110 may be axially inserted into lumen 137 of port housing 134 relative to the longitudinal axis (L). Inner elongate member 110 may be configured to move (e.g., axially) relative to port housing 134. During movement, port housing 134 may remain fluidly connected to inner elongate member 110 such that port 135 may receive and/or deliver fluid into and/or from inner elongate member 110. The axial length of inner elongate member 110 that is positioned within port housing 134 may be greater than the maximum distance of travel of inner elongate member 110 during expansion/compression of recanalization device 108.

The handle 106 includes a trigger mechanism 124 including a gripping portion 126 and a linkage 128. As illustrated in FIGS. 1B and 2B-2D, the linkage 128 may be directly or indirectly coupled to inner elongate member 110. Linkage 128 may be directly or indirectly coupled to adapter 142 and adapter 142 may be directly or indirectly coupled to inner elongate member 110. Adapter 142 may define lumen 132 which extends longitudinally between the proximal end and distal end of adapter 142. Lumen 132 may directly or indirectly engage with inner elongate member 110 such that adapter 142 is coupled with inner elongate member 110. For example, inner elongate member 110 may include and/or define a component/feature that engages with a portion of lumen 132. As shown in FIGS. 2D and 2F, inner elongate member 110 includes a collar 145 that is coupled with lumen 132 such that inner elongate member 110 and adapter 142 axially translate in unison. In one example, collar 145 may be fixedly attached (e.g., crimped, glued, welded, and the like) to the outer surface of inner elongate member 110 and the collar 145 may be fixedly attached (e.g., crimped, glued, welded, and the like) to lumen 132 of adapter 142. Lumen 132 may define a tapered configuration such that the diameter of the tapered portion is equal to or less than the outer diameter of inner elongate member 110. The tapered portion of adapter 142 may engage (“grip”) outer surface of inner elongate member 110.

Adapter 142 may further define cavity 133 which may be oriented axially relative to longitudinal axis (L) and lumen 132. Cavity 133 may extend circumferentially around lumen 132 of adapter 142. Adapter 142 may be configured to directly or indirectly engage with a biasing member 127. Biasing member 127 may include, but is not limited to, a spring (e.g., compression spring, conical spring, disk spring, extension spring), a piston, rubber, an elastic band, and combinations thereof. In one example, cavity 133 may be configured to directly or indirectly engage with a biasing member 127. Biasing member 127 may extend between cavity 133 of adapter 142 and an inner surface of housing 130, actuator 136 and/or leadscrew 140. For example, biasing member 127 may extend between cavity 133 of adapter 142 in the direction of distal portion 131A of housing 130.

Adapter 142 may be directly or indirectly coupled to outer surface of inner elongate member 110. Linkage 128 may be configured to convert the movement of gripping portion 126 of trigger mechanism into axial movement of adapter 142. For example, linkage 128 may be configured to convert the rotational movement of gripping portion 126 into axial movement of adapter 142 along the longitudinal axis (L). When actuated by gripping portion 126 of trigger mechanism 124, linkage 128 advances adapter 142 longitudinally in the direction of the distal end 104 of system 100. Adapter 142 is coupled with inner elongate member 110 such that movement of adapter causes, at least in part, inner elongate member 110 to move. In some instances, inner elongate member 110 may move relative to middle elongate member 112.

The operation of the recanalization device 108 will be discussed in more detail below with regard to FIGS. 9A and 9B. When actuated, movement of the trigger mechanism causes inner elongate member 110 to advance such that the distal portion 111A of the inner elongate member 110 is displaced (e.g., relative to the distal portion 113A of the middle elongate member 112) from an initial position. In one embodiment, the gripping portion 126 of the trigger mechanism 124 moves to actuate linkage 128. Actuation of linkage 128 thereby urges the adapter 142 from its stationary position and causes adapter 142 to move axially along longitudinal axis (L). Axial movement of adapter 142 causes the distal portion 111A of the inner elongate member 110 to advance (e.g., relative to the distal portion 113A of the middle elongate member 112) from an initial position. Because the distal portion 111A of the inner elongate member 110 is coupled to the distal portion 109A of the recanalization device 108, the recanalization device 108 is contracted from an expanded position as the distal portion 111A of the inner elongate member 110 is advanced. That is, the inner elongate member 110 advances while the middle elongate member 112 remains relatively stationary. However, it should be understood that movement of middle elongate member 112 is possible and such movement does not depart from the spirit/scope of this disclosure. When the gripping portion 126 is released by the user, the biasing member 127 forces the actuator 142 and the linkage 128 to revert to a non-actuated position and thus causes the inner elongate member 110 to advance axially towards the proximal portion 102. The distal portion 111A of the inner elongate member 110 then returns to the initial position, and the recanalization device 108 returns to the same expanded position before the linkage 128 was activated.

The trigger mechanism 124 allows a user to easily and efficiently navigate through the vasculature of a patient by allowing the recanalization device 108 to be easily adjusted between a first position and a second position (e.g., contracted and returned to its initial position). Specifically, because the recanalization device 108 is able to be contracted with a grip of the trigger mechanism 124 and returned to its original position as soon as the grip is released, the recanalization device 108 can be advanced or retracted through a constricted and dimensionally variable area in the vasculature of the patient without the user having to manually expand the recanalization device 108 after the navigation. Moreover, the controllability of the recanalization device 108 is superior over recanalization systems that are reactionary when traveling through constricted areas. Said differently, recanalization systems that are incapable of adjustment by a user simply rely on their deformability to travel through a constricted area. At least one benefit of using the controllable recanalization device 108 is that the recanalization device 108 is less likely to damage the vasculature as the device travels through a constricted area within the vessel. Additionally, the system described herein enables a user to control the diameter of the recanalization device 108 and advance/retract the recanalization device 108 in a contracted state. Yet another potential benefit is that a user may not require an angiogram to determine the diameter of the recanalization device 108 after it has been contracted. Yet another potential benefit is that a user may actuate the recanalization device 108 to grab/engage with obstructions on the vessel wall to liberate the obstruction from the wall.

Although the trigger mechanism 124 of the illustrated embodiment includes linkage 128, gripping portion, and actuator 142, other mechanisms may be utilized to advance and retract inner elongate member 110, as described herein. In other embodiments, the trigger mechanism 124 may be a shifter knob, button, pull pin, and/or other motion actuating mechanism. Further, in other embodiments, the trigger mechanism 126 may be coupled to the proximal portion 113B of the middle elongate member 112 instead of the proximal portion 111B of the inner elongate member 110.

As shown in FIGS. 2D and 2E, recanalization system 100 may also enable diameter adjustment of the recanalization device 108. The handle 106 further includes an actuator 136, an indication scale 138 indicating a relative expansion amount of the recanalization device 108 (as described further herein), and a leadscrew 140. The leadscrew 140 is coupled to the proximal portion 113B of the middle elongate member 112. The leadscrew 140 may have a threaded outer surface configured to couple or mate with a threaded inner surface of the actuator 136. The actuator 136 extends out of the housing 130 from one or more openings 131 therein (e.g., a pair of openings 131 on opposing sides of the housing 130) such that the actuator 136 is accessible outside the housing 130 by a user. The actuator 136 is configured to threadably engage the leadscrew 140 over an entire range of movement to expand/contract the recanalization device 108. In the illustrated embodiment, the actuator 136 is a rotatable knob.

In some embodiments, leadscrew 140 defines an indicator (e.g., a tabbed feature) 139 which may extend outwardly and be positioned relative to indication scale 138. Indicator 139 may extend outwardly from leadscrew 140 in a direction perpendicular to longitudinal axis (L). In some instances, indicator 139 may extend outwardly and circumferentially (or semi-circumferentially) around leadscrew 140. In other instances, indicator 139 may be directly or indirectly mounted with respect to leadscrew 140. In operation, as leadscrew 140 axially advances along the longitudinal axis (L) either in the distal direction or proximal direction, indicator 139 may move axially in the direction which corresponds with the leadscrew 140. The indicator 139 may correlate with indication scale 138 to indicate a relative expansion amount of the recanalization device 108. The indication scale 138 may include features/elements to depict the expansion of the recanalization device 108 in a scale format, for example, using alphanumeric characters, symbols, and combinations thereof. The features/elements may be situated such that the indicator 139 is positioned at a point along the indication scale 138 which corresponds to the relative expansion of the recanalization device 108.

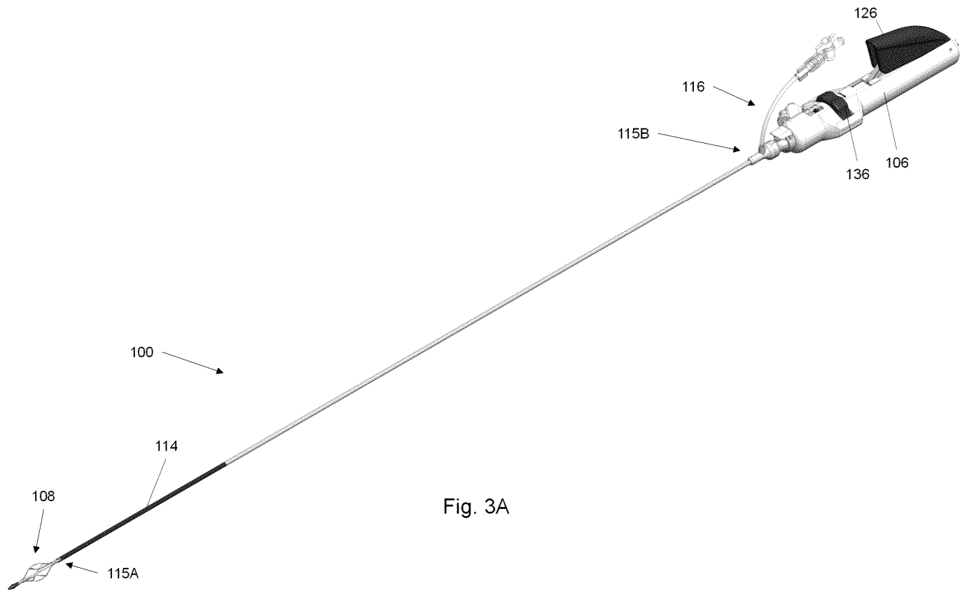

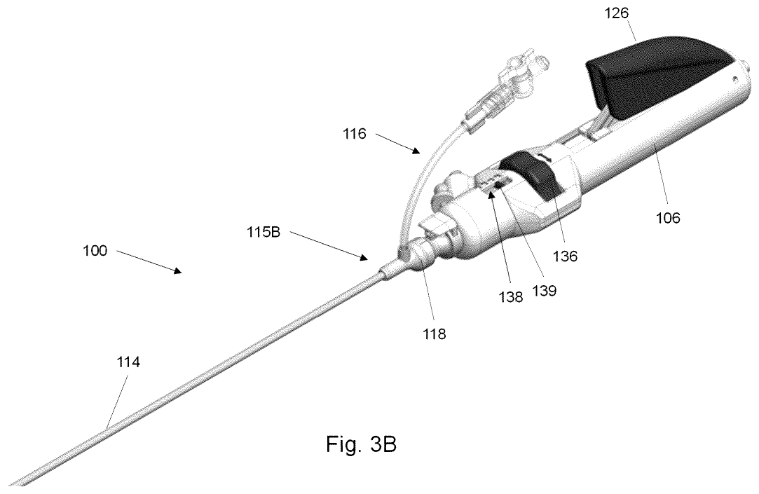

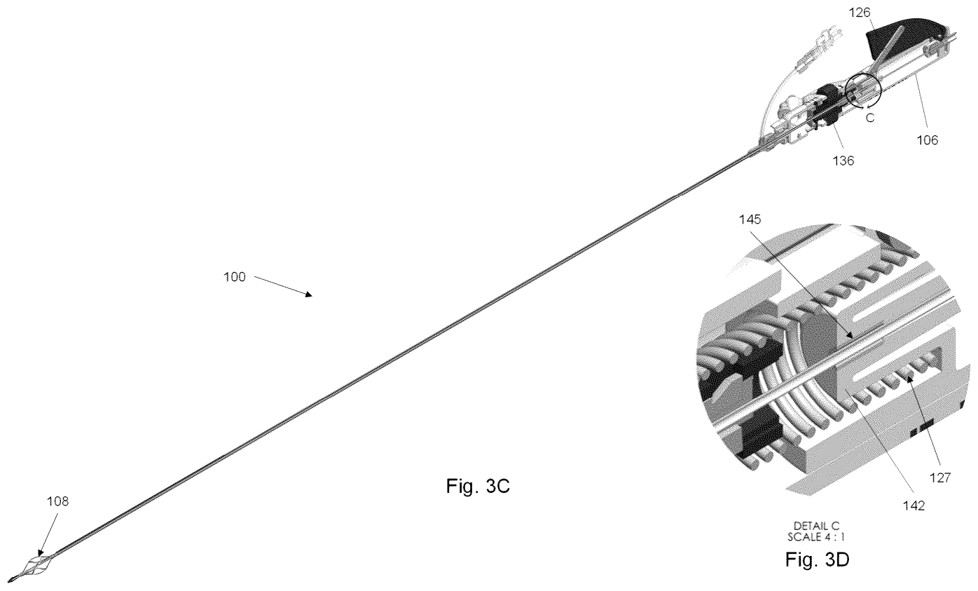

Rotation of the actuator 136 relative to the housing 130 (e.g., by the user) can drive the leadscrew 140 to translate proximally and/or distally (e.g., between the distal and proximal portions 131A, 131B of the housing 130) to thereby drive the coupled middle elongate member 112 to translate relative to the inner elongate member 110. This relative movement of the elongate members 110, 112 increases/decreases the length of the recanalization device 108 to respectively, radially contract/expand the recanalization device 108, as described in further detail below. For example, during radial expansion of the recanalization device 108, the proximal end 109B of the recanalization device 108 may be fixed or nearly fixed (e.g., so as to allow minimal movement) in position relative to the housing 130 and the distal end 109A of the recanalization device 108 may move in the direction of the proximal end 109B such that the distance that separates the distal end 109A and the proximal end 109B of the recanalization device 108 decreases from X1 to X2, as shown in FIGS. 9A and 9B, respectively. During radial contraction of the recanalization device 108, the proximal end 109B of the recanalization device 108 may be fixed or nearly fixed (e.g., so as to allow minimal movement) in position relative to the housing 130 and the distal end 109A of the recanalization device 108 may move in the direction away from the proximal end 109B such that the distance that separates the distal end 109A and the proximal end 109B of the recanalization device 108 increases from X2 to X1, as shown in FIGS. 9B and 9A, respectively. The extent of the collapsing and expanding of recanalization device 108 may be monitored using the display provided by indication scale 138 for viewing by a user (as shown in FIGS. 1A and 3B).

In some embodiments, recanalization system 100 may include sealing cap 156. Sealing cap 156 may be mounted with respect to indicator 139 of leadscrew 140, either directly or indirectly. Thus, the sealing cap 156 may be distal to leadscrew 140. In some embodiments, the handle 106 may include flush ports, openings, lumens, and/or the like. In the illustrated embodiment, the handle 106 includes a flush port 146 for the middle elongate member 112. Flush port 146 may be in fluid communication with at least sealing cap 156. Flush port 146 may be in fluid communication with sealing cap 156 and at least one hole 158 of middle elongate member 112. Flush port 146 may be configured to remove air from middle elongate member 112 through the at least one hole 158 and sealing cap 156.

Middle elongate member 112 may further define at least one hole 158 extending through the middle elongate member 112 (as shown in FIGS. 2D and 2E). For example, the at least one hole 158 extends through an outer wall of the middle elongate member 112. In some instances, middle elongate member 112 may include a plurality of holes 158. Holes 158 may be located at specific longitudinal positions along the length of the middle elongate member. At each longitudinal position the holes may be further spaced circumferentially about the middle elongate member 112. For example, holes 158 may be positioned in groups and each group may be spaced between about 90 degrees to about 120 degrees around the circumference of the middle elongate member 112. Each section may include about three holes, although more or less is acceptable. Holes 158 may be configured to evacuate air from the middle elongate member 112. For example, evacuate air prior to introduction into a patient. Holes 158 may evacuate air into distal inner cavity 143 of leadscrew 140. Distal inner cavity 143 may be in fluid communication with sealing cap 156.

Leadscrew 140 may further define blocking member 152, elongate member retention device 150 and sealing device 154, each may be positioned within proximal inner cavity 141 and axial to longitudinal axis (L). Sealing device 154 may restrict fluids from traveling between distal inner cavity 143 and proximal inner cavity 141. In some instances, sealing device 154 may be positioned between blocking member 152 and elongate member retention device 150. Sealing device 154 may be an o-ring, a gasket, or the like. Sealing device 154 may define a through hole sizably configured to allow inner elongate member 110, middle elongate member 112 or inner elongate member 110 and middle elongate member 112 to extend therethrough while still maintaining sealing properties between distal inner cavity 143 and proximal inner cavity 141.

Elongate member retention device 150 may be configured to directly or indirectly interface with one or more elongate members 110, 112, 114. Elongate member retention device may include a longitudinal hole such that one or more elongate members 110, 112, 114 extends therethrough. In some instances, inner elongate member 110 may extend through the longitudinal hole. Elongate member retention device 150 is configured to directly/indirectly engage with blocking member 152 and sealing device 154. Elongate member retention device 150 may be configured to retain blocking member 152 and sealing device 154 in position relative to adapter 140.

A blocking member 152 may be included along the length of the middle elongate member 112. Blocking member 152 may be configured to restrict axial movement, rotational movement, or axial movement and rotational movement of the middle elongate member 112. Blocking member 152 may be made integral with, and outwardly project from, the middle elongate member 112. In some instances, blocking member 152 may be mounted relative to the middle elongate member 112. For example, blocking member 152 may be fixedly attached to the middle elongate member 112. Blocking member 152 may be shaped to at least partially engage with inner cavity 141 so as to limit movement of the middle elongate member 112. Blocking member 152 may be positioned within a corresponding recessed feature of leadscrew 140 such that movement of blocking member 152 and middle elongate member 112 is restricted. Movement of middle elongate member 112 may be restricted to axial movement that corresponds to movement of leadscrew 140. Blocking member 152 may include a through hole such that at least inner elongate member 110 may extend therethrough.

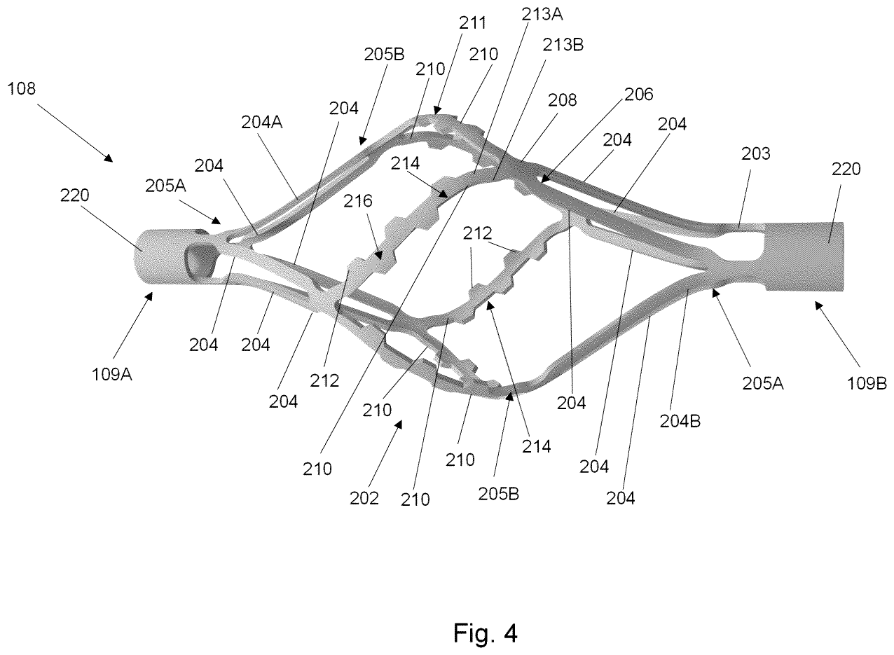

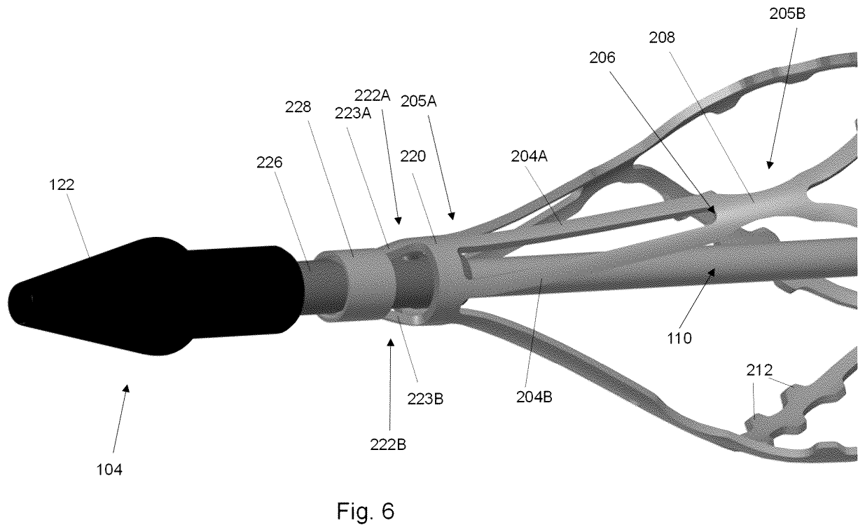

Referring to FIGS. 4 and 5A, the recanalization device 108 includes a center 202 between the distal and proximal ends 109A, 109B of the recanalization device. Although the term center 202 is used, it should be recognized that center 202 may be located at any position between distal and proximal ends that enables the functionality of the recanalization device 108. The recanalization device 108 includes a plurality of support beams or struts 204 extending from respective distal and proximal ends 109A, 109B toward the center 202. In the illustrated embodiment, three pairs of support struts 204 extend from the distal end 109A of the recanalization device 108 toward the center 202 of the recanalization device 108, and three pairs of support struts 204 extend from the proximal end 109B of the recanalization device 108 to the center 202 of the recanalization device 108. More specifically, in the illustrated embodiment, the support struts 204 are generally similar or identical to one another and each extend from a first end 205A proximate either distal end 109A or proximal end 109B of the recanalization device 108 to a second end 205B proximate the center 202 of the recanalization device 108. Each pair of struts 204 is made integral with the respective first or second end 205A, 205B by a rib 203. The struts 204A and 204B are shown most clearly in FIGS. 5B and 6. The reference numbers 204A and 204B are used to identify specific struts 204 in FIGS. 4-6 for purposes of describing the present illustrated embodiment. It should be understood that although the depicted recanalization device 108 includes support struts 204 that are generally similar or identical to one another, the support struts 204 may be differently sized compared to one another, without departing from the spirit/scope of this disclosure. It should also be understood that other struts 204, not specifically identified by 204A or 204B, are similar to the identified struts 204A, 204B. Accordingly, unless otherwise indicated, the description related to struts 204A, 204B also applies to the other struts 204 of the recanalization device 108. As a result, reference number 204 will continue to be used to refer to the struts 204.

Each pair of adjacent support struts 204 is connected to a single junction 206 which connects to an apex 208 of a plurality of diamond cell struts 210. More specifically, the junction 206 is proximate the second ends 205B of the support struts 204, and the diamond cell struts 210 extend between (i) the second ends 205B of the support struts 204 that extend proximally from the distal end 109A of the recanalization device 108 and (ii) the second ends 205B of the support struts 204 that extend distally from the proximal end 109B of the recanalization device 108.

Adjacent support struts 204, for example adjacent support struts 204A, 204B, may be positioned in a parallel configuration such that support strut 204A is parallel with support strut 204B. In another embodiment and as illustrated in the figures, support strut 204A and support strut 204B may be in a non-parallel configuration. Specifically, support struts 204A and 204B may depict a triangular configuration. In one example, support struts 204A and 204B may be further apart in a triangular configuration at first end 205A and may be closer together at second end 205B. In another example, support struts 204A and 204B may be further apart in a triangular configuration at second end 205B and may be closer together at first end 205A. The triangular configuration of support struts 204A and 204B may provide increased rigidity and torque resistance to recanalization device 108.

In the illustrated embodiment, the pairs of support struts 204 provide more support, as compared to support from a single support strut configuration, to the center 202 of the recanalization device 108 and to the diamond cell struts 210 because the pairs of support struts 204 are geometrically larger and therefore increase the second moment of inertia than is possible for a single strut configuration. That is, by pairing the support struts 204, the pair of support struts 204 provide greater support to the recanalization device 108 than a single strut configuration due to the increased in cross-sectional area offered by each pair of struts versus the cross-sectional area offered by a single strut. The pairs of support struts 204 also increase torque resistance of the recanalization device 108, as compared to a single strut configuration. While six pairs of support struts 204 and six diamond cell struts 210 are shown in the illustrated embodiment, the recanalization device 108 may include additional or fewer pairs of support and diamond cell struts 204, 210 in other embodiments. Further, in other embodiments, the recanalization device 108 may include a plurality of support struts 204 and diamond cell struts 210 with a single or plurality of support struts (e.g., single, double, triple, etc.). Each diamond cell strut 210 is made integral with two apex members 208, and each diamond cell strut 210 has a highpoint 211 between the associated apex members 208. The highpoint 211 is located a greater radial distance from longitudinal axis L, shown in FIG. 4, than the associated apex members 208.

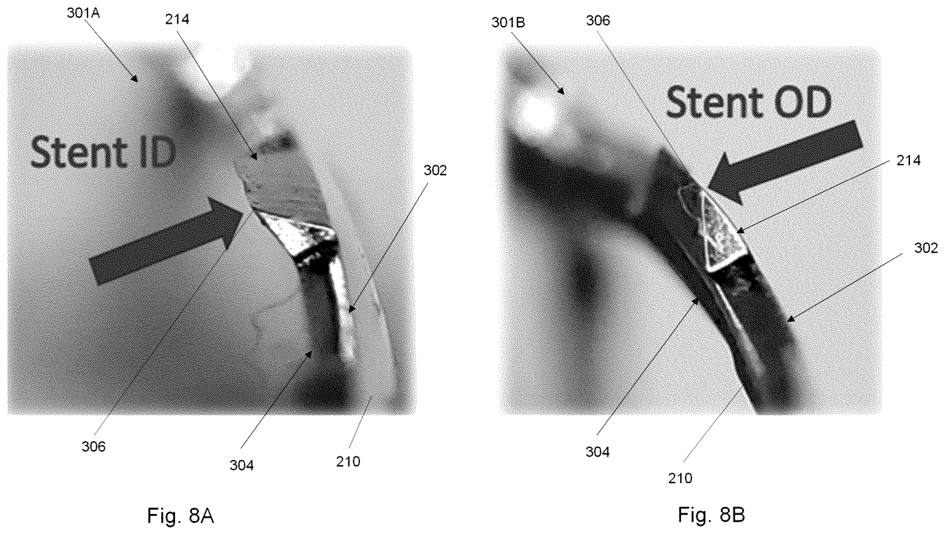

In the illustrated embodiment, each of the diamond cell struts 210 include a plurality of teeth 212 and edge modifications 214. The teeth 212 and edge modifications 214 (described in more detail with respect to FIGS. 8A and 8B) are configured to engage with the clot material within the vasculature of a patient. The teeth 212 are arranged in an alternating pattern. In another embodiment, the teeth 212 may be arranged in a sequential pattern. In the exemplary embodiment, some teeth 212 extend outwardly from a first edge 213A of diamond cell struts 210, and other teeth 212 extend outwardly from a second edge 213B of the diamond cell struts 210. In an exemplary embodiment, the teeth 212 extend along a length of the diamond cell struts 210. The teeth 212 along each edge 213A, 213B are spaced apart. The teeth 212 along the second edge 213B are aligned laterally with the spaces that separate adjacent teeth 212 along the first edge 213A. The teeth 212 are configured to add more surface area to a surface 216 of the diamond cell struts 210 and create an uneven surface along the surface 216 such that the recanalization device 108 has more opportunities to engage with the clot material of the vasculature of the patient. Because the teeth 212 and edge modifications 214 are features that are added to the diamond cell struts 210, the teeth 212 and edge modifications 214 do not negatively impact the radial force of the recanalization device 108. In the illustrated embodiment, the teeth 212 are sized and shaped as trapezoids. In other embodiments, the teeth 212 may be sized and shaped as any other suitable shape including, for example, triangles, semi-circles, etc.

Recanalization device 108 may define collar 220. Collar 220 may be connected to one or more struts 204, either directly or indirectly, at the distal portion 113A, at the proximal portion 113B, or at the distal portion 113A and the proximal portion 113B. Collar 220 may have a cross-sectional shape, referring to a shape viewed perpendicular to longitudinal axis (L), that is cylindrical, quadrilateral, triangular, and combinations thereof. Collar 220 may include and/or define, either directly or indirectly, features and/or components that extend outwardly, inwardly, or outwardly and inwardly from and/or along the longitudinal axis (L). Collar 220 may be semi-permanently or permanently attached to one or more of elongate members 110, 112, 114, tip 122, extension member 226, and combinations thereof. Collar 220 may be attached via adhesive (e.g., glue bonds), welding, fasteners, crimping and/or the like.

Recanalization device 108 may define one or more engagement members 222. Specifically, collar 220 of recanalization device 108 may define one or more engagement members 222. Engagement members 222 may extend laterally from collar 220 towards distal end 104. In some instances, and as illustrated in FIGS. 5A-7B, recanalization device 108 may define at least two engagement members 222. The reference numbers 222A and 222B are used to identify specific engagement members 222 in FIGS. 5A-7B for purposes of describing the present illustrated embodiment. It should be understood, however, that engagement members 222A and 222B may be collectively referred to as 222. Engagement member 222A may oppose engagement member 222B on either side of longitudinal axis (L). Engagement members 222 may be configured to directly or indirectly engage with at least one of elongate members 110, 112, 114, tip 122, extension member 226, and combinations thereof. In one embodiment, engagement members 222 may be configured to removably engage with a corresponding feature/element of extension member 226. In some instances, engagement members 222 may be fixedly attached to extension member 226 in a permanent or semi-permanent configuration. However, recanalization device 108 may be configured to expand and contract between a first position and a second position, as discussed herein.

One or more engagement members 222 may each define one or more tabs 224 and one or more tab arms 223. Engagement member 222A may define tab arm 223A extending outwardly from collar 220 towards distal end 104. Distal portion of tab arm 223A may define tab 224A. Engagement member 222B may define tab arm 223B extending outwardly from collar 220 towards distal end 104. Distal portion of tab arm 223B may define tab 224B. The reference numbers 223A and 223B are used to identify specific tab arms 223 in FIGS. 5A-7B for purposes of describing the present illustrated embodiment. It should be understood, however, that tab arms 223A and 223B may be collectively referred to as 223. Similarly, the reference numbers 224A and 224B are used to identify specific tabs 224 in FIGS. 5A-7B for purposes of describing the present illustrated embodiment. It should be understood, however, that tabs 224A and 224B may be collectively referred to as 224.

Tab arms 223A and 223B may extend outwardly from collar 220 in the direction of the distal end 104 in a parallel, semi-parallel, and/or non-parallel configuration relative to the other tab arm and/or longitudinal axis (L). In some instances, tab arms 223A and 223B may extend outwardly from collar 220 in the direction of the distal end in a tapered configuration such that as the tab arms 223A and 223B extend in the generally longitudinal direction from the collar 220 the ends of the tab arms 223A and 223B are separated by radial distances that are less than the radial distances that separate the tab arms 223A and 223B where they are made integral with the collar 220. Tab arms 223A and 223B may be biased inwards towards longitudinal axis (L).

Tab arms 223A and 223B may each define tab 224 near the distal end of each tab arm 223. Specifically, tab arm 223A may define tab 224A near the distal end thereof and tab arm 223B may define tab 224B near the distal end thereof. Tabs 224A and 224B may be configured to directly or indirectly engage with at least one of elongate members 110, 112, 114, tip 122, extension member 226, and combinations thereof. In one embodiment, tabs 224 may define one or more features that directly or indirectly engage with extension member 226. In another embodiment, tabs 224 may include one or more elements that directly or indirectly engage with extension member 226. For example, extension member 226 may define corresponding openings (not shown) to directly or indirectly engage with tabs 224. The corresponding openings may extend at least partially through a portion of the sidewall of extension member 226. The corresponding openings may also extend at least partially through the sidewall of extension member 226. The corresponding openings may be “keyed” to tabs 224 such that tabs 224 each fit within the corresponding opening that is configured to receive the tab 224. In some embodiments, tabs 224 may be removably positioned at least partially within corresponding openings of extension member 226. In some instances, tabs 224 may be fixedly attached to extension member 226 in a permanent or semi-permanent configuration.

In some instances, engagement member 222 may define geometry or include elements that include features with a first portion that is narrower (or smaller) in size (e.g., diameter) than a second portion. For example, tab 224 may be larger in width/diameter (e.g., laterally relative to longitudinal axis) than tab arm 223. Thus, tab 224 may engage with “keyed” opening to semi-permanently or permanently restrict movement of recanalization device 108 relative to extension member 226 (or substitute component).

In one embodiment and as illustrated in the Figures (e.g., FIG. 5B), tabs 224 may each define a curved surface in proximity to the longitudinal axis (L). The curved surface of tabs 224A and 224B may each be positioned at least partially within the corresponding opening of extension member 226. The corresponding opening of extension member 226 may prevent (or limit) longitudinal movement, lateral movement, outward movement of engagement member 222, inward movement of engagement member 222, and combinations thereof.

In another embodiment, tabs 224 may each include one or more elements that are configured to at least partially engage with corresponding openings of extension member 226. The corresponding opening of extension member 226 may prevent (or limit) longitudinal movement, lateral movement, outward movement away, inward movement, and combinations thereof. The one or more elements may extend roughly perpendicularly in the direction of the longitudinal axis, parallel to the longitudinal axis, angled relative to the longitudinal axis, and combinations thereof.

It should be understood, however, that tabs 224 may define or include other (or additional) geometry or elements that is/are configured to engage with a corresponding opening of extension member 226, elongate members 110, 112, 114, tip 122, and combinations thereof. Thus, the corresponding openings may be configured to engage with a corresponding tab 224. For example, tab 224 and a corresponding opening may be “keyed”, as noted above. The “keyed” opening and the corresponding tab 224 may each define a shape, or a combination of shapes selected from the group including, but not limited to, circle, triangle, square, star, cross, T-shaped, ellipse, rectangle, pentagon, diamond, trapezoid, hexagon, octagon, and oval. Tab 224 and the “keyed” opening may each define a similar shape or a different shape.

It should also be understood, however, that although some embodiments are discussed with reference to extension member 226, other components of system 100 (e.g., elongate members 110, 112, 114, tip 122) may be substituted for extension member 226, unless their use conflicts with the functionality or design of system 100. Additionally, it should be understood that although engagement member 222 is illustrated near the distal end 109A of recanalization device 108, a similar engagement member 222 may also be positioned near the proximal end 109B of recanalization device 108. In some instances, engagement members 222 may be positioned on the distal end 109A of recanalization device 108 and on the proximal end 109B of recanalization device 108. Moreover, the engagement members 222 positioned on either end 109A, 109B of recanalization device 108 may be differently shaped, sized, positioned, as described herein.

Recanalization device 108 may be removable or partially removable from system 100. Engagement members 222 may be removable or partially removable from the corresponding opening of extension member 226, elongate members 110, 112, 114, tip 122, and combinations thereof. Recanalization device 108 may be removed or partially removed for cleaning, removal of extracted bodily material, and/or changing of recanalization devices 108 of similar or different sizes/shapes.

In another embodiment, system 100 may further include band 228. Band 228 may be configured to directly or indirectly with engagement members 222 to semi-permanently or permanently restrict movement of recanalization device 108 in one or more directions. For example, band 228 may restrict longitudinal movement, lateral movement, outward movement of engagement member 222, inward movement of engagement member 222, and combinations thereof. Bands 228 may define and/or include features and/or elements that restrict movement of recanalization device 108 in one or more directions. In a non-exhaustive list, band 228 may be directly or indirectly constrained with engagement member 222 by using adhesives, welding, fasteners, crimping, press fitting, and combinations thereof.

As illustrated in FIGS. 5A-7B, band 228 may engage directly or indirectly with tabs 224 of engagement member 222 to semi-permanently or permanently restrict movement of recanalization device 108 in one or more directions. In a non-exhaustive list, band 228 may be directly or indirectly constrained with tabs 224 by using adhesives, welding, fasteners, crimping, press fitting, and combinations thereof. Band 228 may be at least semi-rigid to semi-permanently or permanently restrict movement of recanalization device 108 in one or more directions. Band 228 may be one continuous piece which may be slid over (in the longitudinal direction) tabs 224. Band 228 may be a non-continuous single piece which may be clamped (closed) at least partially around tabs 224. Band 228 may be two or more pieces which may at least partially engage around tabs 224 (e.g., in a clamshell configuration). Band 228 may be coaxial to elongate member 110, 112, 114 and/or extension member 226.

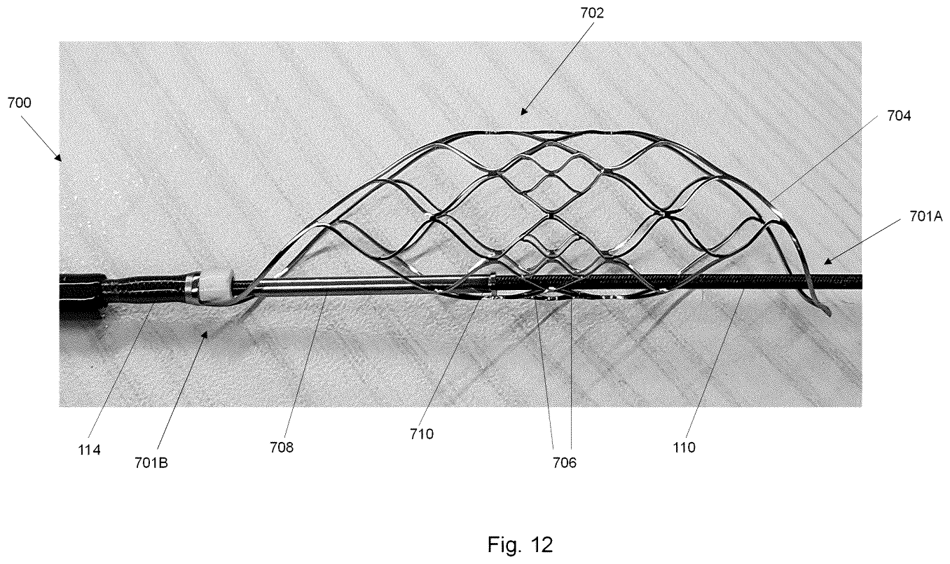

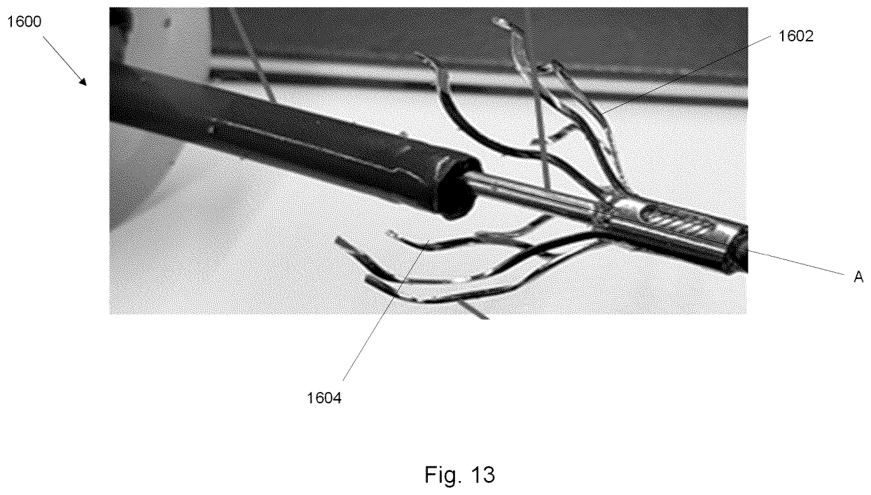

In the illustrated embodiment, the recanalization device 108 is configured as a diamond cell stent pattern. In other embodiments, the recanalization device 108 can be configured differently, as described further herein, especially with respect to FIGS. 12-13.

FIG. 8A illustrates the edge modifications 214 of the diamond cell struts 210 relative to an first edge 301A of the diamond cell struts 210, and FIG. 8B illustrates the edge modifications 214 relative to an second edge 301B of the diamond cell struts 210. The edge modifications 214 allow a reduction in thickness of a portion (e.g. the sharp edge 306) of the diamond cell strut 210 to be reduced, which may make the diamond cell struts 210 more effective at grabbing and/or cutting the clot material included in the vasculature of the patient. In some embodiments, the edge modifications 214 are located along an inner diameter 301A of the teeth 212, an outer diameter 301B of the teeth 212, or an inner diameter 301A and an outer diameter 301B of the teeth 212 (see FIGS. 4 and 5A). In some embodiments, a tooth 212 may have the edge modifications 214 added to the first edge 301A of the tooth 212, and the adjacent teeth 212 may have edge modifications 214 added to the second edge 301B. In other embodiments, the teeth 212 may all have the same edge modifications 214, or the teeth 212 may have any other configuration of edge modifications 214.

The edge modifications 214 of FIGS. 8A and 8B are substantially similar but differ in that in FIG. 8A a sharp edge 306 of the edge modification 214 abuts the first edge first edge 301A. Conversely, in FIG. 8B a sharp edge 306 of the edge modification 214 abuts the second edge second edge 301B. Depending on the location of an edge modification 214 (e.g., whether it abuts the first edge 301A or whether it abuts the second edge 301B). The sharp edge 306 is proximate the first edge 301A or the second edge 301B of the diamond cell struts 210, or, in some embodiments, the sharp edge 306 can be within a thickness of the diamond cell struts 210. The edge modifications 214 may be formed via off-axis laser cutting or post-processing. Further, multiple types of edge modifications 214 (e.g., at the inner diameter 301A, at the second edge 301B, or throughout the thickness of the diamond cell struts) may be included in the same diamond cell strut 210 and/or in the same recanalization device 108 (shown in FIGS. 4-6).

FIGS. 9A and 9B respectively illustrate a first position 402 (e.g., of the recanalization device 108, shown in FIGS. 1A-3D, in a radially expanded position relative to a longitudinal axis (L) and a second position 404 (e.g., of the recanalization device 108, shown in FIGS. 1A-3D, in a radially compressed position relative to longitudinal axis L) of the recanalization device 108 and in comparison to the first position. In the first position 402, the recanalization device 108 has a first diameter D1 and a first length X1. In the second position 404, the recanalization device 108 has a second diameter D2 that is less than the first diameter D1 and a second length X2 that is greater than the first length X1. The diameters D1, D2 are measured from i) an outermost first portion 406 of the diamond cell strut 210 radially furthest from the inner elongate member 110 of the recanalization device 108 to ii) an outermost second portion 408 of the diamond cell strut 210 radially furthest from the inner elongate member 110 in an opposite direction the outermost first portion 406 of the recanalization device 108. The lengths X1, X2 are measured from the distal portion 109A to the proximal portion 109B of the recanalization device 108. In both the first and second positions 402, 404 the recanalization device 108 has been advanced distally out of the outer elongate member 114, shown in FIG. 1A (e.g., past the distal end portion 115A, shown in FIG. 1A, of the outer elongate member 114).

To move the recanalization device 108 into the first position 402, a user can rotate the actuator 136 (as shown in FIGS. 1A-3D) to drive leadscrew 140 either distally or proximally to a desired size (e.g., ratio of diameter to length) of recanalization device 108, as depicted by indication scale 138. For example, movement of leadscrew 140 in the distal direction drives middle elongate member 112 distally over the inner elongate member 110, thereby driving the proximal portion 109B of the recanalization device 108 distally toward the distal portion 109A to radially expand the recanalization device 108 while shortening the recanalization device 108. Movement of leadscrew 140 in the proximal direction drives the middle elongate member 112 proximally over the inner elongate member 110 (which remains stationary or mostly stationary), thereby driving the proximal portion 109B of the recanalization device 108 proximally away from the distal portion 109A to radially compress the recanalization device 108 while increasing the length of the recanalization device 108.

Referring to FIGS. 1A-3D, the indication scale 138 of the handle 106 is configured to provide a visual indication of the extent of radial expansion of the recanalization device 108 in the first position 402. However, the indication scale 138 may also indicate when the recanalization device 108 is in the second position 404, or any other position. In the illustrated embodiment, the indication scale 138 includes an opening for viewing indicator 139 of leadscrew 140. The indication scale 138 may include a scale to show the relative size of recanalization device 108. In other embodiments, the indication scale 138 may be any suitable indicator. Further, although an actuator 136 and leadscrew 140 are described herein as moving the recanalization device 108 into the first position 402, it should be understood that any suitable mechanism can be used to adjust the position of the recanalization device 108.

To move the recanalization device 108 into the second position 404, a user can compress trigger mechanism 124 to extend inner elongate member 110 in the distal direction until recanalization device 108 achieves a desired size (e.g., ratio of diameter to length). Specifically, a user can compress gripping portion 126 of trigger mechanism 124 which translates adapter 142 by way of linkage 128. During compression of gripping portion 126, a user compresses biasing member 127 to longitudinally translate adapter 142, the biasing member 127 is mounted with respect to the distal portion of adapter 142. As noted above, inner elongate member 110 is constrained by adapter 142 and as such, as adapter 142 moves distally, inner elongate member 110 also moves distally, thereby driving the distal portion 109A of recanalization device 108 distally away from the proximal portion 109B to radially compress the recanalization device 108 which lengthening the recanalization device 108. Since distal movement of adapter 142 (and inner elongate member 110) must overcome biasing member 127, a user can compress recanalization device 108 any amount and the recanalization device 108 will maintain that size, which is referred to as the second position 404. Releasing gripping portion 126 entirely will cause the recanalization device 108 to return to the first position 402. In some embodiments, the proximal portion of leadscrew 140 may act as a stop for adapter 142 to ensure the recanalization device 108 is not compressed to a point of damaging the device 108.

The relative movement of the elongate members 110, 112 lengthens (as shown in the second position 404) and/or shortens (as shown in the first position 402) the distance between the distal and proximal ends 109A, 109B of recanalization device 108 to radially expand (as shown in the first position 402) and/or compress (as shown in the second position 404) the recanalization device 108. Although two discrete positions 402, 404 are shown in FIGS. 9A and 9B, the recanalization device can be expanded to any number of continuous positions therebetween, to a position further radially expanded than the first position 402, and/or to a position less radially expanded than the second position 404.

In some embodiments, when the recanalization device 108 is positioned within outer elongate member 114, the recanalization device 108 may be in a compressed position. In some cases, a fully compressed or nearly fully compressed position. Upon distal movement of recanalization device 108 from outer elongate member 114, the recanalization device 108 may remain in the compressed position. Then, a user may adjust actuator 136 to a desired first position 402, as described herein. Thus, a user has complete control over the size of the recanalization device 108 and the positions 402, 404 of the recanalization device 108.

In operation, inner elongate member 110 may move relative to port housing 134 but may maintain fluid connection with port 135. In the first position 402 of recanalization device 108, inner elongate member 110 may be positioned within port housing 134 having a first length. In the second position 404 of recanalization device 108, inner elongate member 110 may be positioned within port housing 134 having a second length. Although the second length of inner elongate member 110 may be shorter than the first length, the second length may be sufficient to maintain fluid connection with port 135.

Recanalization device 108 may be configured to deform when engaged with an obstruction (e.g., clot material within a veinous segment, veinous wall) without lengthening and/or shortening inner elongate member 110 and/or middle elongate member 112. For example, recanalization device 108 may be configured to deform one or more struts 204 and/or diamond cell struts 210 when engaged with an obstruction (e.g., clot material within a veinous segment, veinous wall) without lengthening and/or shortening inner elongate member 110 and/or middle elongate member 112. Said differently, recanalization device 108 may be flexible in order to locally deform in the area engaged with an obstruction, but rigid to clear the obstruction, if necessary. The flexibility of recanalization device 108 is not sufficient to overcome biasing member 127 and move inner elongate member 110 relative to middle elongate member 112.

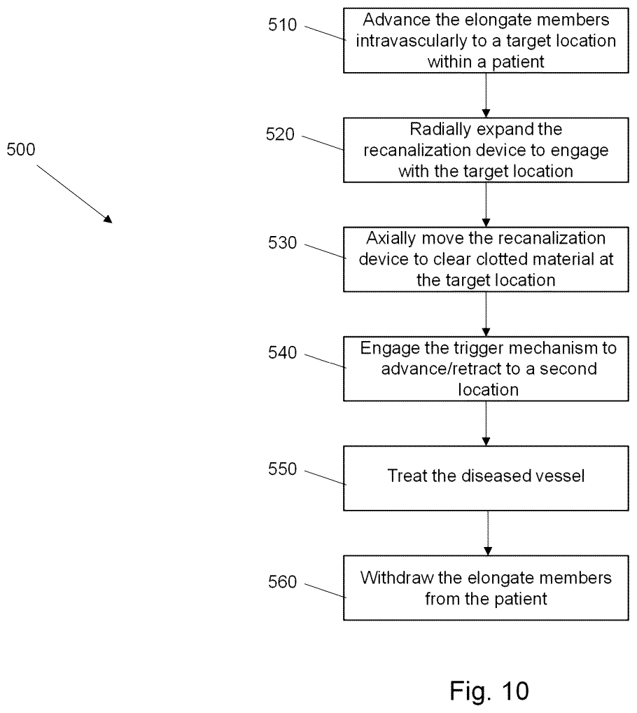

FIG. 10 is a flow chart of a method 500 carried out using the system 100 (shown and described with respect to FIGS. 1A-3D) by a user (e.g., a physician, clinician, etc.). In step 510, the elongate members 110, 112, 114 are advanced intravascularly to a target location (e.g., a veinous segment including chronic clotting material) within a patient. In some instances, the target location may be a position before and/or after a veinous segment including chronic clotting material. Once at the target location, in step 520, the recanalization device 108 is unsheathed and then radially expanded to engage with the vasculature of the target location. Then, in step 530, the recanalization device 108 is axially moved (e.g., in a scrubbing or rotating motion) to debulk the chronic clot material from the target location. Once the chronic clot material is removed from the target location, if necessary in step 540, the trigger mechanism 124 may be engaged to advance and/or retract the elongate members 110, 112, 114 to a second location. At the second location, the recanalization device 108 may treat the diseased vessel in step 550, as described above. During use, if the user engages resistance through an external compression or similar, the trigger mechanism 124 may be engaged to momentarily reduce a diameter of the recanalization device 108 (e.g. reduce a diameter to be less than the first diameter D1). The method 500 ends at step 560 with withdrawing the elongate members from the patient.

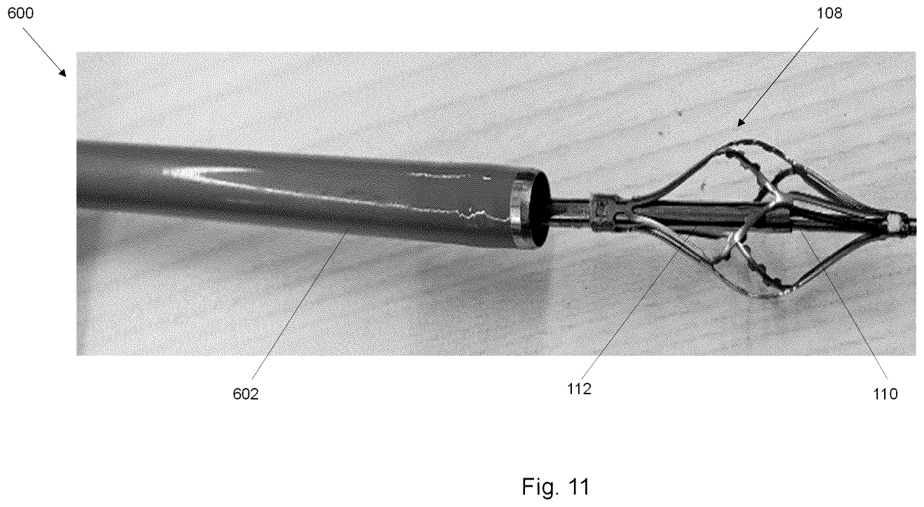

FIG. 11 shows a recanalization system 600. The recanalization system 600 is substantially similar to the recanalization system 100, as shown and described with respect to FIGS. 1A-3D, except that recanalization system 600 includes an outer elongate member 602 that functions differently from the outer elongate member 114 of the recanalization system 100.

Specifically, the outer elongate member 602 is larger in diameter than the outer elongate member 114 and is fitted with a system (not shown) that may be configured to allow for removal of the debulked clot material via aspiration. For example, the debulked clot material may be captured within a lumen of the outer elongate member 602 as the recanalization device 108 debulks the material. That is, the outer elongate member 602 may define an interior pathway within the lumen for the debulked clot material to be flowed from the recanalization device 108. When the elongate members 110, 112, 602 are withdrawn and/or removed from the patient, an aspiration device may be attached to the side port 120, shown in FIGS. 1A-3D, to remove the debulked clot material from the lumen of the outer elongate member 602. In other embodiments, the aspiration of the debulked clot material may occur when the elongate members 110, 112, 602 and recanalization device 108 are still inside and working within the vasculature of the patient.

FIG. 12 and FIG. 13 illustrate different embodiments of the recanalization device 108, as shown and described with respect to FIGS. 1A-9B, of the recanalization system 100, as shown and described with respect to FIGS. 1A-3D.

Referring now to FIG. 12, a recanalization device 700 is shown. The recanalization device 700 is substantially similar to the recanalization device 108, shown and described with respect to FIGS. 1A-9B, except that the recanalization device 700 has a different configuration 702 of struts 704, as described below.

The recanalization device 700 extends from a proximal end 701B to a distal end 701A. The distal end 701A includes an atraumatic shape. In a center (e.g., an area between the distal and proximal ends 701A, 701B) of the recanalization device 700, diamond cell struts 706 are configured to allow for a collection bag (not shown) to be attached to a plurality of diamond cell struts 706. In some embodiments, the collection bag may collect the debulked clot material as the recanalization device 700 works within the vasculature of the patient to lower risk of a thrombotic event happening in the patient. The recanalization device 700 includes a floating shaft 708 that is configured to transmit the force from the middle elongate member 112 (shown, e.g., in FIG. 1A) to the center of the recanalization device 700 and to a weld ring 710. The floating shaft 708 allows the weld ring 710 to move relative to the middle elongate member 112, which allows the recanalization device 700 to be crimped. In other embodiments, the floating shaft 708 can either be coupled to the distal portion 113A of the middle elongate member 112 or to the weld ring 710. Further, in some embodiments, a full ring (now shown) can be coupled to the distal end 701A of the recanalization device 700. These features may increase the pushability of the recanalization device 700 in the vasculature of a patient by transmitting the force from the middle elongate member 112 to the distal end 701A of the recanalization device 700.

FIG. 13 is a perspective view of a recanalization device 1600 including harpoon struts 1602. The harpoon struts 1602 include a metallic structure with arms 1604 that have free ends located away from ends of the harpoon struts 1602 fixed to a distal end of the recanalization device 1600. The free ends are located proximate proximal ends of the recanalization device 1600. In use, the harpoon struts 1602 are positioned within the clot material and are rotated about an axis A to apply tension to the clot. Meanwhile, a second device (not shown) including wall apposition may be advanced or retracted over the harpoon struts 1602 to remove the clot material from the vessel wall. This usage may mimic endovenectomy and how tension is applied to the clot material prior to cutting the clot material away. Similarly, the harpoon struts 1602 are configured to apply tension prior to using another device to radially cut the clot away from the vessel wall. Further, the recanalization device 1600 and harpoon struts 1602 may be used to cut through synechia clots similar to an inverted valvulotome. That is, the recanalization device 1600 may be deployed past the clot and retracted through the lesion. Free ends of the recanalization device 1600 may be shape set to be atraumatic to the vessel wall and to limit engaging collaterals. As the device 1600 is retracted, the arms 1604 may get engage the clot, and, as the clot moves toward a distal end of the recanalization device 1600, the arms 1604 may engage with a sharpened edge at a base of the arms 1604.

The following clauses further define particular aspects and embodiments of the present disclosure.

Clause 1. A recanalization system including a handle located at a proximal end of the system, a first elongate member, and a second elongate member. The first and second elongate members being movable relative to each other elongate member, and a recanalization device located at a distal end of the system. The recanalization device including, a first end portion coupled to the first elongate member, a second end portion coupled to the second elongate member, and at least one strut extending between the first and second end portions. The recanalization device is configured to be (i) selectively radially expanded and compressed and (ii) axially moved relative to the first and second elongate members.

Clause 2. The recanalization system of clause 1, wherein the axial movement of the recanalization device is at least one of (i) axial translation back and forth or scrubbing and (ii) rotating.

Clause 3. The recanalization system according to any of the proceeding clauses, wherein the first and second elongate members are catheters, wherein the catheters are hypotubes, and wherein the hypotubes are formed of nitinol.

Clause 4. The recanalization system according to any of the proceeding clauses 1, wherein the recanalization device further includes a third elongate member, wherein the first and second elongate members are included within a lumen in the third elongate member, and wherein the first and second elongate members are moveable relative to the third elongate member.

Clause 5. The recanalization system of clause 4, wherein the third elongate member is an aspirational catheter, a delivery catheter, or a guide catheter, and wherein the third elongate member includes a braid or coil reflow construction.

Clause 6. The recanalization system according to any of the proceeding clauses, wherein the handle includes an actuator, an indication scale, and a leadscrew rotatably coupled to the actuator, and wherein the actuator and the leadscrew are configured to move the second elongate member relative to the first elongate member.

Clause 7. The recanalization system according to any of the proceeding clauses, wherein a proximal end of the second elongate member is coupled to the leadscrew, and wherein the leadscrew is activated by the actuator.

Clause 8. The recanalization system according to any of the proceeding clauses, wherein the leadscrew defines an indicator that is configured to move relative to an indication scale of the handle to visually display an extent of radial expansion of the at least one strut of the recanalization device.

Clause 9. The recanalization system according to any of the proceeding clauses, wherein the handle further includes a trigger mechanism coupled to the first elongate member, wherein, when actuated, the trigger mechanism is configured to advance the recanalization device from a first position to a second position and thereby radially contract the recanalization device from an expanded configuration.

Clause 10. The recanalization system of clause 9, wherein, when released, a biasing member coupled to the trigger mechanism is configured to return the first elongate member to the first position and thereby expand the recanalization device to the expanded configuration.

Clause 11. The recanalization system according to any of the proceeding clauses, wherein the recanalization device is a stent.

Clause 12. The recanalization system according to any of the proceeding clauses, wherein the recanalization device further includes a third portion extending between the first end portion and the second end portion.

Clause 13. The recanalization system of clause 12, wherein the at least one strut of the recanalization device includes a plurality of pairs of support struts, wherein a first subset of the plurality extend from the first end portion toward the third portion, and wherein a second subset of the plurality extend from the second end portion toward the third portion.

Clause 14. The recanalization system of clause 13, wherein the plurality of pairs of support struts include six pairs of support struts, and wherein the first subset includes three pairs of support struts, and wherein the second subset includes three pairs of support struts.

Clause 15. The recanalization system according to any of clauses 12-14, wherein the at least one strut of the recanalization device further includes a plurality of diamond cell struts extending between the support struts at the third portion of the recanalization device.

Clause 16. The recanalization system of clause 15, wherein the plurality of diamond cell struts includes six diamond cell struts.

Clause 17. The recanalization system according to any of clauses 15-16, wherein each pair of support struts is connected to a single junction that connects to an apex of each diamond cell strut.

Clause 18. The recanalization system according to any of clauses 15-17, wherein each of the diamond cell struts include a plurality of teeth along a length of the diamond cell struts, and wherein each of the diamond cell struts include two edges.

Clause 19. The recanalization system of clause 18, wherein the teeth are sized and shaped as at least one of a trapezoid, a triangle, and a semi-circle.

Clause 20. The recanalization system according to any of clauses 18-19, wherein the edges of each diamond cell strut at least partially include an edge modification, wherein the edge modification includes a reduced thickness of the edge terminating in a sharp edge.

Clause 21. The recanalization system according to any of the proceeding clauses, wherein a collection bag is included within the recanalization device and extends from the first end portion to the second end portion.

Clause 22. The recanalization system according to any of the proceeding clauses, wherein the first and second elongate members are included within and moveable relative to an aspiration device.

Clause 23. The recanalization system according to any of the proceeding clauses, wherein the recanalization device further includes two opposing tabs that extend outwardly from the first end portion or the second end portion of the recanalization device, the two opposing tabs are configured to be biased inward towards each other, wherein the two opposing tabs are configured to engage with at least one of the first elongate member or the second elongate member, either directly or indirectly.

Clause 24. The recanalization system of clause 23 further including a band configured to engage with the two opposing tabs of the recanalization device, wherein the band is configured to restrict movement of the two opposing tabs of the recanalization device.

Clause 25. A method for recanalization including (i) advancing a plurality of elongate members of a recanalization system intravascularly to a target location within a patient, (ii) radially expanding a recanalization device of the recanalization system to engage with the target location, (iii) axially moving the recanalization device to debulk clotted material at the target location, and (iv) withdrawing the plurality of elongate members of the recanalization system from the patient.

Clause 26. The method of clause 25 further including (i) engaging a trigger mechanism of the recanalization system to advance or retract the plurality of elongate members to a second location, and (ii) debulking clotted material from the second location.

Clause 27. A recanalization device including a first end and a second end, and a center between the first and second ends, the first and second ends being spaced apart along an axis, the first and second ends being movable toward and away from the center of the recanalization device, at least one strut member extending away from each of the first and second ends, and terminating at an apex member, a diamond cell strut extending between and integral with two apex members, each diamond cell strut including opposed outwardly directed edges, and a number of teeth located along each edge.

Clause 28. The recanalization device of clause 27, wherein the diamond cell strut members include a plurality of teeth along each of the opposed edges.

Clause 29. The recanalization device of clause 28, wherein the plurality of teeth along the one edge are spaced apart, and wherein each of the teeth along the second edge are aligned laterally with a space separating adjacent teeth along the one edge.

Clause 30. The recanalization device according to any of clauses 27-29, wherein the diamond cell strut members include three teeth along one edge and two teeth along the opposite edge.

Clause 31. The recanalization device according to any of clauses 27-30, wherein the recanalization device includes a plurality of strut members extending away from each of the first and second ends.

Clause 32. The recanalization device of clause 31, wherein the recanalization device includes six strut members extending away from each of the first and second ends.

Clause 33. The recanalization device according to any of clauses 27-32, wherein the recanalization device includes three pairs of strut members extending from each of the first and second ends, and wherein each pair of struts terminates at an apex member.

Clause 34. The recanalization device according to any of clauses 27-33, wherein the teeth terminate in a cutting edge.

Clause 35. The recanalization device according to any of clauses 27-34, wherein each of the teeth has a trapezoidally-shaped adjustment surface.

Clause 36. The recanalization device according to any of clauses 27-35, wherein each of the diamond cell strut members has a highpoint that is located a greater distance from the recanalization device axis that the diamond cell strut ends integral with the apex members.

Clause 37. The recanalization device according to any of clauses 27-36 further including two opposing tabs that extend outwardly from the first end portion or the second end portion, the two opposing tabs are configured to be biased inward towards each other.

The above detailed descriptions of embodiments of the disclosure are not intended to be exhaustive or to limit the disclosure to the precise form disclosed above. Although specific embodiments of, and examples for, the disclosure are described above for illustrative purposes, various equivalent modifications are possible within the scope of the disclosure, as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the disclosure have been described herein for purposes of illustration, but well-known structures and functions have not been shown or described in detail to avoid unnecessarily obscuring the description of the embodiments of the disclosure. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (i) any single item in the list, (ii) all of the items in the list, or (iii) any combination of the items in the list. Additionally, the terms “including” and “comprising” are used throughout to mean including at least the recited feature(s) such that any greater number of the same feature and/or additional types of other features are not precluded. It will also be appreciated that specific embodiments have been described herein for purposes of illustration, but that various modifications may be made without deviating from the disclosure. Further, while advantages associated with some embodiments of the disclosure have been described in the context of those embodiments, other embodiments may also exhibit such advantages, and not all embodiments need necessarily exhibit such advantages to fall within the scope of the disclosure. Accordingly, the disclosure may encompass other embodiments not expressly shown or described herein.

What is claimed is:

1. A recanalization system comprising:
a handle located at a proximal end of the system;
a first elongate member;
a second elongate member, the first and second elongate members being movable relative to each other elongate member; and
a recanalization device located at a distal end of the system, the recanalization device comprising:
a first end portion coupled to the first elongate member;
a second end portion coupled to the second elongate member; and
at least one strut extending between the first and second end portions;
wherein the recanalization device is configured to be (i) selectively radially expanded and compressed and (ii) axially moved relative to the first and second elongate members.

2. The recanalization system of claim 1, wherein the axial movement of the recanalization device is at least one of (i) axial translation back and forth or scrubbing and (ii) rotating.

3. The recanalization system of claim 1, wherein the first and second elongate members are catheters, wherein the catheters are hypotubes, and wherein the hypotubes are formed of nitinol.

4. The recanalization system of claim 1, wherein the recanalization device further comprises a third elongate member, wherein the first and second elongate members are included within a lumen in the third elongate member, and wherein the first and second elongate members are moveable relative to the third elongate member.

5. The recanalization system of claim 4, wherein the third elongate member is an aspirational catheter, a delivery catheter, or a guide catheter, and wherein the third elongate member includes a braid or coil reflow construction.

6. The recanalization system of claim 1, wherein the handle includes an actuator, an indication scale, and a leadscrew rotatably coupled to the actuator, and wherein the actuator and the leadscrew are configured to move the second elongate member relative to the first elongate member.

7. The recanalization system of claim 6, wherein a proximal end of the second elongate member is coupled to the leadscrew, and wherein the leadscrew is activated by the actuator.

8. The recanalization system of claim 7, wherein the leadscrew defines an indicator that is configured to move relative to the indication scale to visually display an extent of radial expansion of the at least one strut of the recanalization device.

9. The recanalization system of claim 1, wherein the handle further comprises a trigger mechanism coupled to the first elongate member, wherein, when actuated, the trigger mechanism is configured to advance the recanalization device from a first position to a second position and thereby radially contract the recanalization device from an expanded configuration.

10. The recanalization system of claim 9, wherein, when released, a biasing member coupled to the trigger mechanism is configured to return the first elongate member to the first position and thereby expand the recanalization device to the expanded configuration.

11. The recanalization system of claim 1, wherein the recanalization device is a stent.

12. The recanalization system of claim 1, wherein the recanalization device further comprises a third portion extending between the first end portion and the second end portion.

13. The recanalization system of claim 12, wherein the at least one strut of the recanalization device includes a plurality of pairs of support struts, wherein a first subset of the plurality extend from the first end portion toward the third portion, and wherein a second subset of the plurality extend from the second end portion toward the third portion.

14. The recanalization system of claim 13, wherein the plurality of pairs of support struts include six pairs of support struts, and wherein the first subset includes three pairs of support struts, and wherein the second subset includes three pairs of support struts.

15. The recanalization system of claim 14, wherein the at least one strut of the recanalization device further includes a plurality of diamond cell struts extending between the support struts at the third portion of the recanalization device.

16. The recanalization system of claim 15, wherein the plurality of diamond cell struts includes six diamond cell struts.

17. The recanalization system of claim 16, wherein each pair of support struts is connected to a single junction that connects to an apex of each diamond cell strut.

18. The recanalization system of claim 15, wherein each of the diamond cell struts include a plurality of teeth along a length of the diamond cell struts, and wherein each of the diamond cell struts include two edges.

19. The recanalization system of claim 18, wherein the teeth are sized and shaped as at least one of a trapezoid, a triangle, and a semi-circle.

20. The recanalization system of claim 18, wherein the edges of each diamond cell strut at least partially include an edge modification, wherein the edge modification includes a reduced thickness of the edge terminating in a sharp edge.

21. The recanalization system of claim 1, wherein a collection bag is included within the recanalization device and extends from the first end portion to the second end portion.

22. The recanalization system of claim 1, wherein the first and second elongate members are included within and moveable relative to an aspiration device.

23. The recanalization system of claim 1, wherein the recanalization device further comprises two opposing tabs that extend outwardly from the first end portion or the second end portion of the recanalization device, the two opposing tabs are configured to be biased inward towards each other, wherein the two opposing tabs are configured to engage with at least one of the first elongate member or the second elongate member, either directly or indirectly.

24. The recanalization system of claim 23 further comprising a band configured to engage with the two opposing tabs of the recanalization device, wherein the band is configured to restrict movement of the two opposing tabs of the recanalization device.

25. A method for recanalization comprising:
advancing a plurality of elongate members of a recanalization system intravascularly to a target location within a patient;
radially expanding a recanalization device of the recanalization system to engage with the target location;
axially moving the recanalization device to debulk clotted material at the target location; and
withdrawing the plurality of elongate members of the recanalization system from the patient.

26. The method of claim 25 further comprising:
engaging a trigger mechanism of the recanalization system to advance or retract the plurality of elongate members to a second location; and
debulking clotted material from the second location.

27. A recanalization device comprising:
a first end and a second end, and a center between the first and second ends, the first and second ends being spaced apart along an axis, the first and second ends being movable toward and away from the center of the recanalization device;
at least one strut member extending away from each of the first and second ends, and terminating at an apex member;
a diamond cell strut extending between and integral with two apex members, each diamond cell strut comprising:
opposed outwardly directed edges; and
a number of teeth located along each edge.

28. The recanalization device of claim 27, wherein the diamond cell strut members include a plurality of teeth along each of the opposed edges.

29. The recanalization device of claim 28, wherein the plurality of teeth along the one edge are spaced apart, and wherein each of the teeth along the second edge are aligned laterally with a space separating adjacent teeth along the one edge.

30. The recanalization device of claim 29, wherein the diamond cell strut members include three teeth along one edge and two teeth along the opposite edge.

31. The recanalization device of claim 27, wherein the recanalization device includes a plurality of strut members extending away from each of the first and second ends.

32. The recanalization device of claim 31, wherein the recanalization device includes six strut members extending away from each of the first and second ends.

33. The recanalization device of claim 30, wherein the recanalization device includes three pairs of strut members extending from each of the first and second ends, and wherein each pair of struts terminates at an apex member.

34. The recanalization device of claim 30, wherein the teeth terminate in a cutting edge.

35. The recanalization device of claim 34, wherein each of the teeth has a trapezoidally-shaped adjustment surface.

36. The recanalization device of claim 27, wherein each of the diamond cell strut members has a highpoint that is located a greater distance from the recanalization device axis that the diamond cell strut ends integral with the apex members.

37. The recanalization device of claim 27 further comprising two opposing tabs that extend outwardly from the first end portion or the second end portion, the two opposing tabs are configured to be biased inward towards each other.
